(19) United States
(12) Patent Application Publication
(10) Pub. No.: US 2024/0371467 A1
(43) Pub. Date: Nov. 7, 2024

(54) METHYLATION BIOMARKER SELECTION APPARATUSES AND METHODS

(71) Applicant: ACT Genomics (IP) Limited, Shatin (HK)

(72) Inventors: Tun-Wen PAI, Taipei City (TW); Yi-Hsuan LAI, Taipei City (TW); Shu-Jen CHEN, Taipei City (TW); Fang-Cheng SU, Taipei City (TW)

(73) Assignee: ACT Genomics (IP) Limited, Shatin (HK)

(21) Appl. No.: 18/293,413

(22) PCT Filed: Sep. 22, 2022
(86) PCT No.: PCT/IB2022/058985

§ 371 Date: Jan. 30, 2024

Related U.S. Application Data
(60) Provisional application No. 63/261,780, filed on Sep. 28, 2021.

(51) Int. Cl. G16B 20/20; G16B 40/20; G16H 50/20

(52) U.S. Cl. CPC G16B 20/20; G16B 40/20; G16H 50/20

(57) ABSTRACT

Methylation biomarker selection apparatuses and methods are provided. A methylation biomarker selection apparatus stores a plurality of first data sets and a plurality of second data sets, wherein each of the first data sets includes a plurality of methylation degrees corresponding to a plurality of methylation loci, and each of the second data sets includes at least one medical record. The methylation biomarker selection apparatus determines a plurality of primary biomarkers by identifying a plurality of differentiable loci from the methylation loci according to the methylation degrees, determines a plurality of secondary biomarkers by identifying a plurality of comorbidities of a target disease, and associated genes thereof based on the second data sets, and determines a plurality of candidate biomarkers based on a correlation analysis of the primary biomarkers and the secondary biomarkers.

### PRIORITY

This application claims priority to U.S. Provisional Patent Application No. 63/261,780 filed on Sep. 28, 2021, which is hereby incorporated by reference in its entirety.

### FIELD OF THE INVENTION

The present invention relates to methylation biomarker selection apparatuses and methods. More specifically, the present invention relates to methylation biomarker selection apparatuses and methods that provide biomarkers pertaining to a target disease based on comorbidity analysis.

### BACKGROUND OF THE INVENTION

Biomarkers have played an important role in the medical field, such as for diagnosing diseases and developing drugs. An ideal biomarker for a target disease should be of high sensitivity and high specificity so that the target disease can be detected in an early stage and prognosis can be evaluated. The common approach to discover biomarker(s) pertaining to a target disease is to investigate into the samples of the patients with the target disease. However, as the samples analyzed by the common approach are quite limited in terms of both quantity and diversity, the results are usually unsatisfactory (e.g., the derived biomarker(s) is/are without high sensitivity and/or without high specificity) and insufficient (e.g., only few biomarkers are derived).

Consequently, a technique that can provide a sufficient amount of biomarkers that are highly sensitive and highly specific to a target disease is still needed.

### SUMMARY OF THE INVENTION

An objective of this invention is to provide a methylation biomarker selection apparatus. The methylation biomarker selection apparatus comprises a storage and a processor, wherein the processor is electrically connected to the storage. The storage is configured to store a plurality of first data sets, wherein each of the first data sets comprises a plurality of methylation degrees corresponding to a plurality of methylation loci. The storage is also configured to store a plurality of second data sets, wherein each of the second data sets comprises at least one medical record. The processor is configured to perform the following operations: (a) determining a plurality of primary biomarkers by identifying a plurality of differentiable loci from the methylation loci according to the methylation degrees, (b) determining a plurality of secondary biomarkers by identifying a plurality of comorbidities of a target disease, and associated genes thereof based on the second data sets, and (c) determining a plurality of candidate biomarkers based on a correlation analysis of the primary biomarkers and the secondary biomarkers.

Another objective of this invention is to provide a methylation biomarker selection method for use in an electronic apparatus. The electronic apparatus stores a plurality of first data sets, wherein each of the first data sets comprises a plurality of methylation degrees corresponding to a plurality of methylation loci. The electronic apparatus also stores a plurality of second data sets, wherein each of the second data sets comprises at least one medical record. The methylation biomarker selection method comprises the following steps: (a) determining a plurality of primary biomarkers by identifying a plurality of differentiable loci from the methylation loci according to the methylation degrees, (b) determining a plurality of secondary biomarkers by identifying a plurality of comorbidities of a target disease, and associated genes thereof based on the second data sets, and (c) determining a plurality of candidate biomarkers based on a correlation analysis of the primary biomarkers and the secondary biomarkers.

The methylation biomarker selection technique (at least comprises the methylation biomarker selection apparatuses and methods) provided by the present invention utilizes two different kinds of data sets (i.e., the first data sets and the second data sets) to discover candidate biomarkers pertaining to a target disease. While the first data sets comprise methylation degrees of various methylation loci, the second data sets comprise medical record(s). With the first data sets, differentiable loci can be identified as the primary biomarkers pertaining to the target disease. With the second data sets, comorbidities of the target disease, and associated genes thereof can be identified so as to provide the secondary biomarkers pertaining the target disease. As both methylation degrees and comorbidities of the target disease are considered, the methylation biomarker selection technique of the present invention can provide candidate biomarkers that are highly sensitive and highly specific to the target disease. Furthermore, as the candidate biomarkers are determined based on a correlation analysis of the primary biomarkers and the secondary biomarkers, a sufficient amount of candidate biomarkers can be provided.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 illustrates the schematic view of a methylation biomarker selection apparatus 1 in some embodiments of the present invention.

FIG. 2 illustrates the general data process flow for finding out the candidate biomarkers based on methylation degrees and comorbidities associated with a target disease.

FIG. 3 illustrates the data process flow for deriving the first data sets D1_1, . . . , D1_q in some embodiments of the present invention.

FIG. 4 illustrates the data process flow for weight calculation and target biomarker selection in some embodiments of the present invention.

FIG. 5 illustrates the schematic view of an exemplary recurrent neural network used in some embodiments of the present invention.

FIG. 6 illustrates the main flowchart of a methylation biomarker selection method in some embodiments of the present invention.

FIG. 7 illustrates the main flowchart of a methylation biomarker selection method in some embodiments of the present invention.

FIG. 8 illustrates the main flowchart of the step S709 in some embodiments of the present invention.

FIG. 9 shows an exemplary result of clinical validation of the target biomarkers.

### DETAILED DESCRIPTION

In the following descriptions, the methylation biomarker selection apparatuses and methods of the present invention will be explained regarding certain embodiments thereof. However, these embodiments are not intended to limit the present invention to any specific environment, application, or implementations described in these embodiments. Therefore, descriptions of these embodiments are to provide illustration rather than to limit the scope of the present invention. It should be noted that, in the following embodiments and the attached drawings, elements unrelated to the present invention are omitted from depiction. In addition, dimensions of elements and any dimensional scales between individual elements in the attached drawings are provided only for ease of depiction and illustration but not to limit the scope of the present invention.

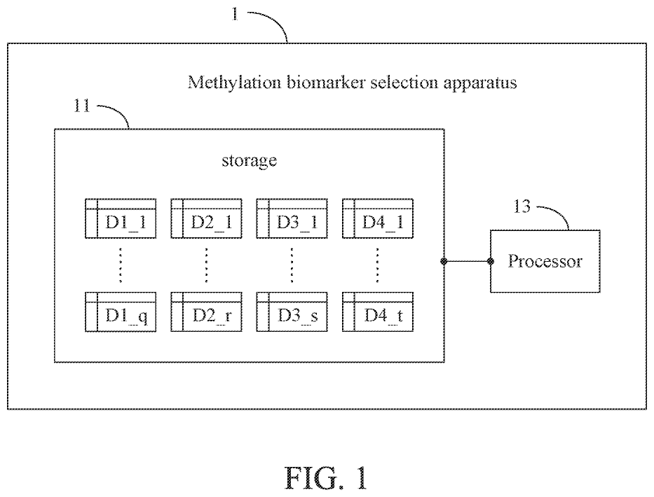

FIG. 1 illustrates the schematic view of a methylation biomarker selection apparatus 1 in some embodiments of the present invention. The methylation biomarker selection apparatus 1 comprises a storage 11 and a processor 13, wherein the storage 11 is electrically connected to the processor 13. The storage 11 may be a memory, a Universal Serial Bus (USB) disk, a portable disk, a Hard Disk Drive (HDD), or any other non-transitory storage media, apparatus, or circuit that can store data and known to a person having ordinary skill in the art. The processor 13 may be one of the various processors, central processing units (CPUs), microprocessor units (MPUs), digital signal processors (DSPs), or other computing apparatuses known to a person having ordinary skill in the art.

The storage 11 stores a plurality of first data sets D1_1, . . . , D1_q, wherein each of the first data sets D1_1, . . . , D1_q comprises a plurality of methylation degrees corresponding to a plurality of methylation loci. Please note that a methylation locus is a locus of gene that refers to CG rich or CG poor DNA region that includes at least one differentially methylated region. In some embodiments, methylation locus comprises CpG methylation locus and non-CpG methylation locus. In addition, the storage 11 stores a plurality of second data sets D2_1, . . . , D2_r, wherein each of the second data sets D2_1, . . . , D2_r comprises at least one medical record.

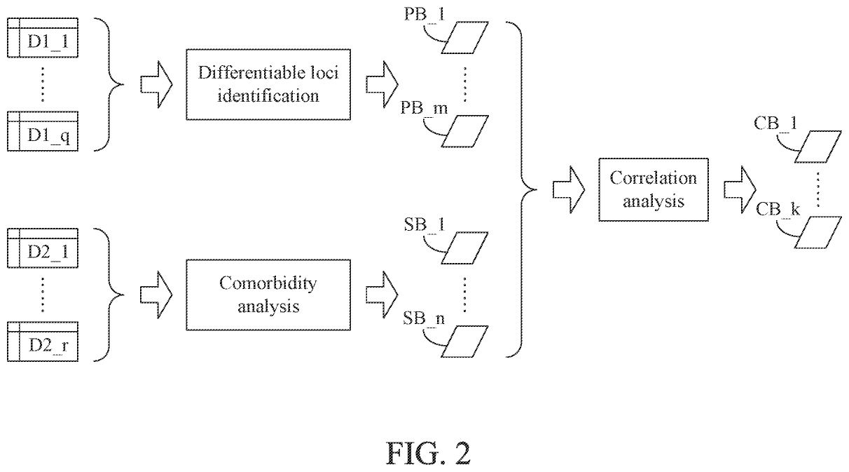

The methylation biomarker selection apparatus 1 aims to find out biomarkers that may be highly related to a target disease based on methylation degrees and comorbidities associated with the target disease, and the general data process flow of which is illustrated in FIG. 2. Specifically, the processor 13 determines a plurality of primary biomarkers PB_1, . . . , PB_m by identifying a plurality of differentiable loci from the methylation loci recorded in the first data sets D1_1, . . . , D1_q according to the methylation degrees recorded in the first data sets D1_1, . . . , D1_q, determines a plurality of secondary biomarkers SB_1, . . . , SB_n by identifying a plurality of comorbidities of a target disease, and associated genes thereof based on the second data sets D2_1, . . . , D2_r, and determines a plurality of candidate biomarkers CB_1, . . . , CB_k based on a correlation analysis of the primary biomarkers PB_1, . . . , PB_m and the secondary biomarkers SB_1, . . . , SB_n. The candidate biomarkers CB_1, . . . , CB_k are the biomarkers that may be highly related to the target disease so that they may be used for further investigation and/or evaluation of the target disease. As used herein, “comorbidity” refers to one or more conditions, syndromes, diseases, or disorders that causes, is caused by, or co-occur with the target disease and can be either directly or indirectly linked to the target disease. In some embodiments, the first data sets D1_1, . . . , D1_q are generated by the methylation array or methylation sequencing. In some embodiments, the target disease includes but not limited to brain cancer, breast cancer, colon cancer, endocrine gland cancer, esophageal cancer, female reproductive organ cancer, head and neck cancer, hepatobiliary system cancer, kidney cancer, lung cancer, mesenchymal cell neoplasm, prostate cancer, skin cancer, stomach cancer, tumor of exocrine pancreas and urinary system cancer.

The detailed descriptions of the first data sets D1_1, . . . , D1_q, the second data sets D2_1, . . . , D2_r, and the operations performed by the processor 13 in various embodiments are provided below.

### First Data Sets

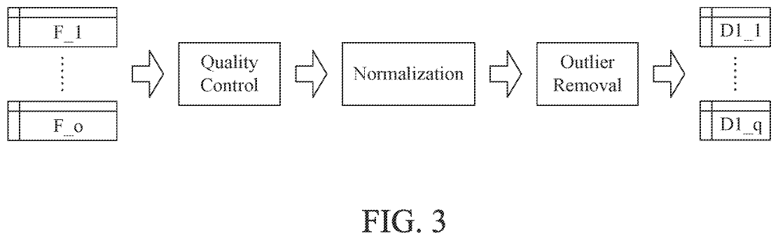

In some embodiments, the methylation biomarker selection apparatus 1 derives the first data sets D1_1, . . . , D1_q from the data files generated by the methylation array (e.g., Illumina Infinium HumanMethylation450 BeadChip (450K Chip)), and the data process flow of which is illustrated in FIG. 3. In those embodiments, the methylation biomarker selection apparatus 1 is installed with the Chip Analysis Methylation Pipeline (ChAMP) package, and the processor 13 imports the data files F_1, . . . , F_o (e.g., the IDAT files) of the methylation array from a first database (e.g., The Cancer Genome Atlas (TCGA)) through a transceiving interface (not shown) of the methylation biomarker selection apparatus 1. Each of the imported data files F_1, . . . , F_o comprises a plurality of methylation degrees corresponding to a plurality of methylation loci (e.g., N methylation degrees correspond to N methylation loci one to one, and N is a positive integer greater than one). In the data files F_1, . . . , F_o generated by the methylation array, a methylation degree is called a 3 value. Then, the processor 13 may derive the first data sets D1_1, . . . , D1_q by pre-processing the imported data files F_1, . . . , F_o, which usually involves quality control, normalization, and outlier removal.

An example regarding quality control is given herein. In this example, probes that meet any one of the following criteria are excluded: (1) probes with a detection value of P>0.01 in at least one sample, (2) probes with a bead count smaller than 3 in at least 5% of samples, (3) probes targeting non-CpG positions, (4) probes targeting single nucleotide polymorphism (SNP) sites, (5) probes that align to multiple locations, and (6) probes located on X and Y chromosomes. After the aforesaid quality control, only the methylation loci corresponding to the remained probes are kept in the imported data files.

Examples regarding normalization are given herein. The methylation degrees in the aforesaid imported data files are bias because the methylation array adopts two different types of probe design (Inflinium type 1 probe design and Inflinium type 2 probe design); therefore, normalization is required to adjust the biases. For example, beta-mixture quantile normalization (BMIQ), subset-quantile within array normalization (SWAN), peak-based correction (PBC), or Functional normalization (FunNorm) can be used.

An example regarding outlier removal is given herein. The imported data files that have been processed by the aforesaid quality control and normalization are classified into a normal subject group and a disease subject group. The normal subject group comprises the imported data files related to the subjects without the target disease, while the disease subject group comprises the imported data files related to the subjects with the target disease. For each methylation locus in each of the normal subject group and the disease subject group, the outlier(s) are eliminated by the Interquartile Range (IQR) method. A person having ordinary skill in the art shall be familiar with the IQR method and, thus, the details are not given herein. By removing the outliers, the distribution of the methylation degrees of each methylation locus in each of the normal subject group and the disease subject group is in a concentrated form. In this way, noise interferences during primary biomarker selection can be avoided.

The imported data files that have been processed by the aforesaid quality control, normalization, and outlier removal are the first data sets D1_1, . . . , D1_q. Please note that the above examples are not intended to limit the approach for deriving the first data sets D1_1, . . . , D1_q. In some other embodiments, the first data sets D1_1, . . . , D1_q may be derived from other sources and by other approaches as long as each of the first data sets D1_1, . . . , D1_q comprises a plurality of methylation degrees corresponding to a plurality of methylation loci.

### Primary Biomarker Selection

As described above, the processor 13 determines a plurality of primary biomarkers PB_1, . . . , PB_m by identifying a plurality of differentiable loci from the methylation loci recorded in the first data sets D1_1, . . . , D1_q according to the methylation degrees recorded in the first data sets D1_1, . . . , D_q. The differentiable loci are the loci that are more distinguishable among the methylation loci recorded in the first data sets D1_1, . . . , D1_q.

In some embodiments, for each of the methylation loci, the processor 13 determines whether the methylation locus can be selected as a differentiable locus based on an averaged methylation degree difference of the methylation locus and/or a p-value of the methylation locus. The averaged methylation degree difference of a methylation locus reflects the extent that the methylation degrees of the methylation locus from disease subjects are deviated from the methylation degrees of the methylation locus from normal subjects. Specifically, from the methylation loci recorded in the first data sets D1_1, . . . , D1_q, the processor 13 selects the methylation loci having: (i) the averaged methylation degree difference conforming to a first predetermined rule (e.g., the averaged methylation degree difference being greater than a first predetermined threshold) and/or (ii) the p-value conforming to a second predetermined rule (e.g., the p-value being smaller than a second predetermined threshold) as the differentiable loci. The differentiable loci are determined as the primary biomarkers PB_1, . . . , PB_m.

The aforesaid averaged methylation degree difference is elaborated herein. In some embodiments, the first data sets D1_1, . . . , D1_q are classified into a normal subject group and a disease subject group. That is, each first data set in the normal subject group is related to a subject without the target disease, while each first data set in the disease subject group is related to a subject with the target disease. In those embodiments, the processor 13 derives the averaged methylation degree difference of a methylation locus by performing the following operations (a) and (b).

In the operation (a), the processor 13 calculates an averaged normal value according to the methylation degrees corresponding to the methylation locus from the normal subject group. In one example, the averaged normal value is the mean value of the methylation degrees of the methylation locus within the normal subject group, and can be characterized by the following equation (1):

$${β}_{normal \_ avg} = \frac{{\sum }_{i = 1}^{n} {β}_{i}}{n} \qquad ( 1 )$$

In the above equation (1), β_{normal_avg} represents the averaged normal value, βᵢ represents the methylation degree corresponding to the methylation locus from the i^{th} subject in the normal subject group, and n represents the number of subjects in the normal subject group (i.e., the number of the methylation degrees corresponding to the methylation locus in the normal subject group).

In the operation (b), the processor 13 calculates the averaged methylation degree difference according to the averaged normal value and the methylation degrees corresponding to the methylation locus from the disease subject group. In one example, the averaged methylation degree difference is the mean value of a plurality of individual methylation degree differences and can be characterized by the following equation (2):

$$Δβ = \frac{{\sum }_{j = 1}^{m} ( {β}_{j} - {β}_{normal \_ avg} )}{m} \qquad ( 2 )$$

In the above equation (2), Δβ represents the averaged methylation degree difference, βⱼ represents the methylation degree corresponding to the methylation locus from the j^{th} subject in the disease subject group, β_{normal_avg} represents the averaged normal value, and m represents the number of subjects in the disease subject group (i.e., the number of the methylation degrees corresponding to the methylation locus in the disease subject group). In addition, the value (βⱼ−β_{normal_avg}) represents the individual methylation degree differences.

The aforesaid approach for deriving primary biomarkers PB_1, . . . , PB_m has been conducted to various target diseases, and the relevant information and data are listed in Table 1. Please note that the data files from TCGA are of Mar. 15, 2021, and the data files from Gene Expression Omnibus (GEO) database are of Oct. 30, 2021. In Table 1, the variable N_{N} represents the number of the subject without the target disease, and the variable N_{TD} represents the number of the subject with the target disease.

TABLE 1

| | | | | Number of primary |
|---|---|---|---|---|
| Target disease | First database | N_{N}/N_{TD} | Δβ | biomarkers |
| Colorectal cancer | TCGA | 38/314 | 0.5 | 214,088 |
| Lung cancer | TCGA | 42/370 | 0.45 | 320,395 |
| Liver cancer | TCGA | 50/380 | 0.4 | 260,808 |
| Pancreatic cancer | TCGA | 10/185 | 0.35 | 212,524 |
| Prostate cancer | TCGA | 50/503 | 0.45 | 287,206 |
| Breast cancer | TCGA | 50/430 | 0.4 | 297,978 |
| Ovarian cancer | GEO | 7/114 | 0.55 | 123,796 |
| Esophagus cancer | TCGA | 16/186 | 0.45 | 154,709 |
| Stomach cancer | TCGA | 2/395 | 0.35 | 10,470 |

### Second Data Sets

In some embodiments, the methylation biomarker selection apparatus 1 derives the second data sets D2_1, . . . , D2_r from a second database through a transceiving interface (not shown) of the methylation biomarker selection apparatus 1. For example, the second database may be any electronic medical record dataset (e.g., the Taiwan's National Health Insurance Research Database, NHIRD), which comprises a plurality of anonymous electronic medical records (EMRs).

Medical records stored in the second database are related to a plurality of subjects. The subjects with the target disease are selected as an experimental group, while some of the subjects without the target disease are selected as a control group. The subjects in the control group may be randomly selected by matching age groups and genders with five-fold of the subjects in the experimental group. For the control group, medical record(s) of each subject is/are retrieved. For the experimental group, medical record(s) of each subject within a predetermined time interval (e.g., 3, 4 or 5 years before the first diagnosis of the target disease) is/are retrieved. All the retrieved medical records are subjected to data cleaning and integration to yield the second data sets D2_1, . . . , D2_r so that each of the second data sets D2_1, . . . , D2_r corresponds to one subject, and the medical record(s) of the same subject is/are included in one second data set.

Each medical record of the second data sets D2_1, . . . , D2_r has diagnosis information of a subject. If a subject has been diagnosed with one or more diseases, the corresponding medical record(s) will record the diagnosed disease(s). Please note that the present invention does not limit the way to record the diagnosed disease(s). In some embodiments, a diagnosed disease is a specific disease and can be recorded as a disease code followed the International Classification of Diseases (ICD). In some embodiments, a diagnosed disease is a disease group and can be recorded as a disease group code followed the ICD.

In some embodiments, the disease code(s) may be the code(s) from the International Classification of Diseases, Ninth Revision, Clinical Modification (ICD-9-CM). There have been more than 1,000 diseases listed in ICD-9-CM. They contain 17 major chapters as shown in Table 2 and are further classified into various disease groups, which includes several diseases individually. Taking chapter 2 (i.e., neoplasms) of the ICD-9-CM as an example, it has 11 disease groups.

TABLE 2

| Chapter | Disease Name | Code |
|---|---|---|
| 1 | Infectious and Parasitic Diseases | 001-139 |
| 2 | Neoplasms | 140-239 |
| 3 | Endocrine, Nutritional and Metabolic | 240-279 |
| | Diseases, and Immunity Disorders | |
| 4 | Diseases of the Blood and Blood-Forming Organs | 280-289 |
| 5 | Mental Disorders | 290-319 |
| 6 | Diseases of the Nervous System and Sense Organs | 320-389 |
| 7 | Diseases of the Circulatory System | 390-459 |
| 8 | Diseases of the Respiratory System | 460-519 |
| 9 | Diseases of the Digestive System | 520-579 |
| 10 | Diseases of the Genitourinary System | 580-629 |
| 11 | Complications of Pregnancy, Childbirth, and the | 630-679 |
| | Puerperium | |
| 12 | Diseases of the Skin and Subcutaneous Tissue | 680-709 |
| 13 | Diseases of the Musculoskeletal System and | 710-739 |
| | Connective Tissue | |
| 14 | Congenital Anomalies | 740-759 |
| 15 | Certain Conditions Originating in the Perinatal | 760-779 |
| | Period | |
| 16 | Symptoms, Signs, and Ill-Defined Conditions | 780-799 |
| 17 | Injury and Poisoning | 800-999 |

The aforesaid approach for deriving the second data sets D2_1, . . . , D2_r has been conducted to various target diseases, and the relevant information and data are listed in Table 3. Please note that the data sets derived from NHIRD are of Jan. 29, 2016. The disease codes are the code(s) based on ICD-9-CM. In addition, the variable N_{EG} represents the number of the subject in the experimental group, and the variable N_{CG} represents the number of the subject in the control group.

TABLE 3

| Target disease | Second database | N_{EG} | N_{CG} | Disease code(s) |
|---|---|---|---|---|
| Colorectal cancer | NHIRD | 6293 | 30653 | 153/154 |
| Lung cancer | NHIRD | 3351 | 16460 | 162 |
| Liver cancer | NHIRD | 4532 | 21970 | 155 |
| Pancreatic cancer | NHIRD | 637 | 3142 | 157 |
| Prostate cancer | NHIRD | 2310 | 11320 | 185 |
| Breast cancer | NHIRD | 3465 | 17083 | 174 |
| Ovarian cancer | NHIRD | 930 | 4596 | 183 |
| Esophagus cancer | NHIRD | 597 | 2971 | 150 |
| Stomach cancer | NHIRD | 1116 | 5459 | 151 |

### Secondary Biomarker Selection

As described above, the processor 13 determines a plurality of secondary biomarkers SB_1, . . . , SB_n by identifying a plurality of comorbidities of the target disease, and associated genes thereof based on the second data sets D2_1, . . . , D2_r. In some embodiments, the processor 13 identifies a plurality of distinct diagnosed diseases from the second data sets D2_1, . . . , D2_r and determines the secondary biomarkers SB_1, . . . , SB_n by performing the following operations (c), (d), and (e).

In the operation (c), the processor 13 calculates an association degree indicating relevance to the target disease for each of the distinct diagnosed diseases.

In some embodiments, an association degree between a diagnosed disease and the target disease comprises an odds ratio, a p-value, and a supporting rate. For those embodiments, the processor 13 calculates the following four statistical numbers based on the second data sets D2_1, . . . , D2_r: (i) the total number of the subjects with both the diagnosed disease and the target disease, which is represented by the variable N_{DD_DT}, (ii) the total number of the subjects with the diagnosed disease but without the target disease, which is represented by the variable N_{DD_NDT}, (iii) the total number of the subjects without diagnosed disease but with target disease, which is represented by the variable N_{NDD_DT}, and (iv) the total number of the subjects without diagnosed disease and without target disease, which is represented by the variable N_{NDD_NDT}. With the four statistical numbers, the processor 13 can calculate the odds ratio and the supporting rate by the following equations (3) and (4) respectively:

$$Odds Ratio = \frac{{N}_{DD \_ DT} / {N}_{NDD \_ DT}}{{N}_{DD \_ NDT} / {N}_{NDD \_ NDT}} = \frac{{N}_{DD \_ DT} \times {N}_{NDD \_ NDT}}{{N}_{DD \_ NDT} \times {N}_{NDD \_ DT}} \qquad ( 3 )$$

$$Supporting Rate = \frac{{N}_{DD \_ DT} + {N}_{DD \_ NDT}}{{N}_{DD \_ DT} + {N}_{DD \_ NDT} + {N}_{NDD \_ DT} + {N}_{NDD \_ NDT}} \qquad ( 4 )$$

Please note that other indicator that can reflect relevance between two diseases can be used as an association degree. For example, an indicator of relative risk can be used as an association degree in some embodiments.

In the operation (d), among the distinct diagnosed diseases, the processor 13 selects the diagnosed diseases having the association degree conforming to a third predetermined rule as the comorbidities.

For the embodiments that an association degree comprises an odds ratio, a p-value, and a supporting rate, the third predetermined rule comprises three sub-rules for the odds ratio, the p-value, and the supporting rate, respectively. As an example, the three sub-rules may be “the odds ratio being greater than 2,” “the p-value being smaller than 0.05,” and “the supporting rate being greater than 10%.”

In the operation (e), the processor 13 determines a plurality of genes corresponding to the comorbidities as the secondary biomarkers SB_1, . . . , SB_n. For example, the processor 13 may retrieve the genes corresponding to the comorbidities from a third database (e.g., the DisGeNET database, the Online Mendelian Inheritance in Man (OMIM) database) through a transceiving interface (not shown) of the methylation biomarker selection apparatus 1.

The aforesaid approach for deriving the secondary biomarkers SB_1, . . . , SB_n has been conducted to various target diseases under the condition that the third predetermined rule comprises “the odds ratio being greater than 2,” “the p-value being smaller than 0.05,” and “the supporting rate being greater than 10%.” Various significant comorbidities of these target diseases and the relevant data are listed in Table 4 to Table 12. Specifically, Table 4 is for the target disease “colorectal cancer,” Table 5 is for the target disease “lung cancer,” Table 6 is for the target disease “liver cancer,” Table 7 is for the target disease “pancreatic cancer,” Table 8 is for the target disease “prostate cancer,” Table 9 is for the target disease “breast cancer,” Table 10 is for the target disease “ovarian cancer,” Table 11 is for the target disease “esophagus cancer,” and Table 12 is for the target disease “stomach cancer.”

TABLE 4
(Significant comorbidities of colorectal cancer)

| | | | | | | Odd | P- | Number of secondary |
|---|---|---|---|---|---|---|---|---|
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 578 | Gastrointestinal | 717 | 987 | 5576 | 29666 | 3.864899722 | 1.10E−153 | 158 |
| | hemorrhage | | | | | | | |
| 455 | Hemorrhoids | 1257 | 2097 | 5036 | 28556 | 3.398979138 | 3.71E−218 | 33 |
| 564 | Functional digestive | 3027 | 7911 | 3266 | 22742 | 2.664363719 | 8.56E−261 | 804 |
| | disorders not elsewhere | | | | | | | |
| | classified | | | | | | | |
| 532 | Duodenal ulcer | 638 | 1248 | 5655 | 29405 | 2.658242932 | 1.42E−82 | 120 |
| 536 | Disorders of function | 2539 | 7295 | 3754 | 23358 | 2.165602731 | 1.42E−156 | 118 |
| | of stomach | | | | | | | |
| 533 | Peptic ulcer site | 1871 | 5042 | 4422 | 25611 | 2.149209463 | 1.41E−129 | 168 |
| | unspecified | | | | | | | |
| 789 | Other symptoms | 2406 | 7028 | 3887 | 23625 | 2.080755957 | 3.51E−138 | 1025 |
| | involving abdomen | | | | | | | |
| | and pelvis | | | | | | | |

TABLE 5
(Significant comorbidities of lung cancer)

| | | | | | | Odd | P- | Number of secondary |
|---|---|---|---|---|---|---|---|---|
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 486 | Pneumonia, organism | 534 | 748 | 2817 | 15712 | 3.981844379 | 1.88E−116 | 216 |
| | unspecified | | | | | | | |
| 496 | Chronic airway | 458 | 766 | 2893 | 15694 | 3.243559903 | 3.17E−79 | 208 |
| | obstruction, not | | | | | | | |
| | elsewhere classified | | | | | | | |
| 491 | Chronic bronchitis | 879 | 1638 | 2472 | 14822 | 3.217609386 | 8.51E−136 | 299 |
| 490 | Bronchitis, not | 416 | 1031 | 2935 | 15429 | 2.121115604 | 2.11E−34 | 239 |
| | specified as acute | | | | | | | |
| | or chronic | | | | | | | |
| 493 | Asthma | 541 | 1388 | 2810 | 15072 | 2.090606828 | 1.94E−41 | 2205 |

TABLE 6
(Significant comorbidities of liver cancer)

| | | | | | | Odd | P- | Number of secondary |
|---|---|---|---|---|---|---|---|---|
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 571 | Chronic liver disease | 2582 | 2789 | 1950 | 19181 | 9.106350406 | 0 | 649 |
| | and cirrhosis | | | | | | | |
| 70 | Viral hepatitis | 1246 | 1090 | 3286 | 20880 | 7.26364281 | 0 | 1780 |
| 574 | Cholelithiasis | 456 | 665 | 4076 | 21305 | 3.584186177 | 7.59E−91 | 269 |
| 573 | Other disorders of liver | 511 | 757 | 4021 | 21213 | 3.561172734 | 3.52E−100 | 1367 |
| 533 | Peptic ulcer site | 1334 | 3144 | 3198 | 18826 | 2.497772542 | 3.84E−129 | 168 |
| | unspecified | | | | | | | |
| 531 | Gastric ulcer | 576 | 1357 | 3956 | 20613 | 2.211706815 | 2.55E−51 | 153 |

TABLE 7
(Significant comorbidities of pancreatic cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 577 | Diseases of | 91 | 23 | 546 | 3119 | 22.6014 | 3.13E−39 | 763 |
| | pancreas | | | | | | | |
| 574 | Cholelithiasis | 101 | 96 | 536 | 3046 | 5.97882 | 9.49E−33 | 269 |
| 532 | Duodenal ulcer | 101 | 142 | 536 | 3000 | 3.98098 | 1.77E−23 | 120 |
| 571 | Chronic liver | 246 | 434 | 391 | 2708 | 3.9257 | 1.15E−45 | 649 |
| | disease and | | | | | | | |
| | cirrhosis | | | | | | | |
| 211 | Benign neoplasm of | 68 | 95 | 569 | 3047 | 3.83306 | 4.30E−16 | 10169 |
| | other parts of | | | | | | | |
| | digestive system | | | | | | | |
| 533 | Peptic ulcer site | 265 | 532 | 372 | 2610 | 3.49488 | 6.35E−41 | 168 |
| | unspecified | | | | | | | |
| 531 | Gastric ulcer | 129 | 241 | 508 | 2901 | 3.05673 | 7.13E−21 | 153 |

TABLE 8
(Significant comorbidities of prostate cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 600 | Hyperplasia | 1837 | 3542 | 473 | 7778 | 8.52839678 | 0 | 117 |
| | of prostate | | | | | | | |
| 601 | Inflammatory | 350 | 393 | 1960 | 10927 | 4.965012723 | 5.09E−95 | 102 |
| | diseases | | | | | | | |
| | of prostate | | | | | | | |
| 599 | Other disorders | 810 | 1765 | 1500 | 9555 | 2.923342776 | 2.75E−99 | 485 |
| | of urethra and | | | | | | | |
| | urinary tract | | | | | | | |
| 788 | Symptoms involving | 691 | 1624 | 1619 | 9696 | 2.548225049 | 2.51E−70 | 313 |
| | urinary system | | | | | | | |
| 595 | Cystitis | 257 | 591 | 2053 | 10729 | 2.272563036 | 1.37E−25 | 206 |

TABLE 9
(Significant comorbidities of breast cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 217 | Benign neoplasm | 1211 | 747 | 2254 | 16336 | 11.74939094 | 0 | 10171 |
| | of breast | | | | | | | |
| 611 | Other disorders | 1869 | 1895 | 1596 | 15188 | 9.385724205 | 0 | 128 |
| | of breast | | | | | | | |
| 239 | Neoplasms of | 355 | 291 | 3110 | 16792 | 6.586844344 | 1.69E−118 | 10206 |
| | unspecified | | | | | | | |
| | nature | | | | | | | |
| 610 | Benign mammary | 475 | 477 | 2990 | 16606 | 5.530559587 | 2.03E−140 | 174 |
| | dysplasias | | | | | | | |

TABLE 10
(Significant comorbidities of ovarian cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 220 | Benign neoplasm | 337 | 187 | 593 | 4409 | 13.3990405 | 3.20E−145 | 10170 |
| | of ovary | | | | | | | |
| 620 | Noninflammatory | 234 | 170 | 696 | 4426 | 8.753245436 | 1.34E−88 | 5 |
| | disorders of ovary | | | | | | | |
| | fallopian tube and | | | | | | | |
| | broad ligament | | | | | | | |
| 617 | Endometriosis | 138 | 180 | 792 | 4416 | 4.274747475 | 5.57E−34 | 1242 |
| 218 | Uterine leiomyoma | 194 | 398 | 736 | 4198 | 2.78024634 | 2.31E−26 | 10218 |
| 789 | Other symptoms | 512 | 1582 | 418 | 3014 | 2.333621665 | 2.90E−31 | 1025 |
| | involving abdomen | | | | | | | |
| | and pelvis | | | | | | | |
| 614 | Inflammatory disease | 230 | 577 | 700 | 4019 | 2.288611042 | 5.47E−21 | 79 |
| | of ovary fallopian | | | | | | | |
| | tube pelvic cellular | | | | | | | |
| | tissue and peritoneum | | | | | | | |
| 571 | Chronic liver | 123 | 321 | 807 | 4275 | 2.029844005 | 3.44E−10 | 649 |
| | disease and | | | | | | | |
| | cirrhosis | | | | | | | |

TABLE 11
(Significant comorbidities of esophagus cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 733 | Other disorders | 63 | 112 | 534 | 2859 | 3.011587079 | 1.99E−11 | 1203 |
| | of bone and | | | | | | | |
| | cartilage | | | | | | | |
| 627 | Menopausal and | 93 | 210 | 504 | 2761 | 2.426048753 | 3.32E−11 | 10 |
| | postmenopausal | | | | | | | |
| | disorders | | | | | | | |
| 530 | Diseases of | 129 | 309 | 468 | 2662 | 2.374616214 | 9.83E−14 | 1149 |
| | esophagus | | | | | | | |
| 531 | Gastric ulcer | 73 | 170 | 524 | 2801 | 2.29538617 | 1.89E−08 | 153 |
| 571 | Chronic liver | 133 | 371 | 464 | 2600 | 2.008783344 | 6.56E−10 | 649 |
| | disease and | | | | | | | |
| | cirrhosis | | | | | | | |
| 533 | Peptic ulcer site | 151 | 430 | 446 | 2541 | 2.000683074 | 1.17E−10 | 168 |
| | unspecified | | | | | | | |

TABLE 12
(Significant comorbidities of stomach cancer)

| | | | | | | | | Number of |
|---|---|---|---|---|---|---|---|---|
| | | | | | | Odd | P- | secondary |
| Code | Comorbidity | N_{DD}_{_{—}}_{DT} | N_{DD}_{_{—}}_{NDT} | N_{NDD}_{_{—}}_{DT} | N_{NDD}_{_{—}}_{NDT} | Ratio | value | biomarkers |
| 531 | Gastric ulcer | 360 | 444 | 756 | 5015 | 5.378592879 | 5.96E−96 | 153 |
| 578 | Gastrointestinal | 153 | 187 | 963 | 5272 | 4.479184367 | 3.52E−39 | 158 |
| | hemorrhage | | | | | | | |
| 533 | Peptic ulcer site | 527 | 966 | 589 | 4493 | 4.161545167 | 4.14E−93 | 168 |
| | unspecified | | | | | | | |
| 532 | Duodenal ulcer | 157 | 241 | 959 | 5218 | 3.544606891 | 1.76E−31 | 120 |
| 285 | Other and | 136 | 252 | 980 | 5207 | 2.867476514 | 4.98E−21 | 1055 |
| | unspecified | | | | | | | |
| | anemias | | | | | | | |
| 536 | Disorders of | 500 | 1385 | 616 | 4074 | 2.387594355 | 9.24E−38 | 118 |
| | function | | | | | | | |
| | of stomach | | | | | | | |
| 535 | Gastritis | 568 | 1715 | 548 | 3744 | 2.26276521 | 1.47E−34 | 232 |
| | and duodenitis | | | | | | | |

### Candidate Biomarker Selection

After deriving the primary biomarkers PB_1, . . . , PB_m and the secondary biomarkers SB_1, . . . , SB_n, the processor 13 determines a plurality of candidate biomarkers CB_1, . . . , CB_k based on a correlation analysis of the primary biomarkers PB_1, . . . , PB_m and the secondary biomarkers SB_1, . . . , SB_n. In some embodiments, the correlation analysis is intersection or union of the primary biomarker and the secondary biomarker. Please note that different correlation analysis may be used in different embodiments.

As described above, the primary biomarkers PB_1, . . . , PB_m are differentiable loci regarding a target disease, and the secondary biomarkers SB_1, . . . , SB_n are genes corresponding to the comorbidities of the same target disease. Hence, determining the candidate biomarkers CB_1, . . . , CB_k based on a correlation analysis of the primary biomarkers PB_1, . . . , PB_m and the secondary biomarkers SB_1, . . . , SB_n provides a promising result. That is, within the candidate biomarkers CB_1, . . . , CB_k, biomarker(s) that is/are highly sensitive and highly specific to the target disease can be found and can be used for further analysis regarding the target disease.

### Biomarker Functional Clustering

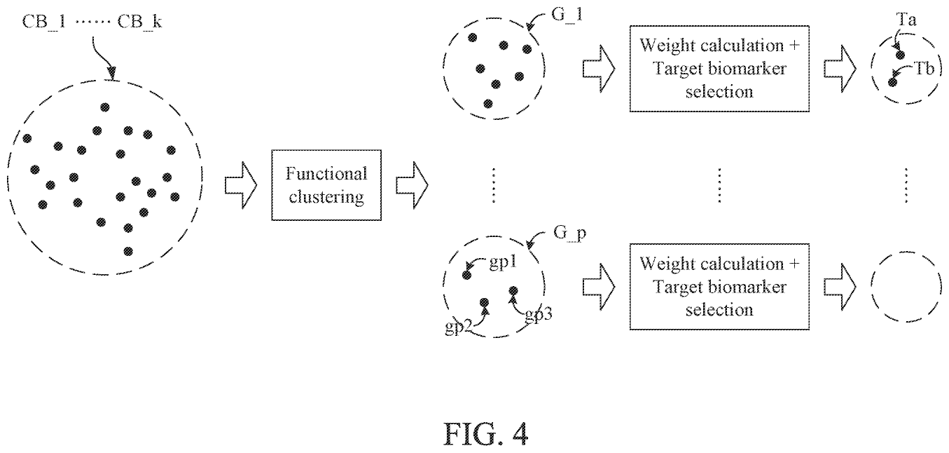

Different candidate biomarkers CB_1, . . . . CB_k represent different functional roles. As shown in FIG. 4, in some embodiments, the processor 13 further clusters the candidate biomarkers CB_1, . . . , CB_k into a plurality of functional clusters G_1, . . . , G_p. In FIG. 4, every black dot represents a candidate biomarker. Candidate biomarkers within the same functional cluster are close to each other in terms of function (e.g., regulating the same function or similar functions).

### Biomarker Functional Clustering Based on Gene Distances

In some embodiments, the processor 13 can cluster the candidate biomarkers CB_1, . . . , CB_k into the functional clusters G_1, . . . , G_p based on a plurality of gene distances between every pair of the candidate biomarkers CB_1, . . . , CB_k. Please note that a gene distance is a value showing the distance in terms of function between two genes.

In some embodiments, the concept of Gene Ontology (GO) is adopted for calculating the gene distances. GO depicts gene functions in a GO tree by a plurality of GO terms, and the GO terms are categorized into three complementary biological concepts including Biological Process (BP), Molecular Function (MF), and Cellular Component (CC). Functions of most human genes are well annotated by GO terms. In those embodiments, each of the candidate biomarkers CB_1, . . . , CB_k is annotated with at least one GO term with reference to a fourth database (e.g., Ensembl Release 104, Ensembl Release 105, Ensembl Release 106 or Ensembl Release 107).

In those embodiments, the processor 13 calculates a gene distance for every pair of the candidate biomarkers CB_1, . . . , CB_k. Specifically, the processor 13 can calculate a gene distance between a first candidate biomarker and a second candidate biomarker by the following operations (f) and (g).

In the operation (f), the processor 13 calculates a GO term distance for each of at least one GO term pair between the first candidate biomarker and the second candidate biomarker. Please note that a GO term distance is a value showing the distance (in terms of function) between two GO terms.

A concrete example is given herein for better understanding. In this example, the first candidate biomarker is the gene “B3GNTL1” and is annotated with a GO term “GO:0016757,” while the second candidate biomarker is the gene “PLD5” and is annotated with three GO terms “GO:0003824,” “GO:0008152,” and “GO:0016021.” Three GO term pairs can be formed between the first candidate biomarker and the second candidate biomarker, including (GO:0016757, GO:0003824), (GO:0016757, GO:0008152), and (GO:0016757, GO:0016021). The processor 13 calculates a GO term distance for each of the three GO term pairs.

In the operation (g), the processor 13 determines the gene distance between the first candidate biomarker and the second candidate biomarker according to the GO term distance(s) derived in the operation (f). In some embodiments, the processor 13 takes the mean value of the GO term distance(s) as the gene distance between the first candidate biomarker and the second candidate biomarker.

The above concrete example is continued herein for better understanding. For the first candidate biomarker “B3GNTL1” and the second candidate biomarker “PLD5,” the GO term distances of the three GO term pairs (GO:0016757, GO:0003824), (GO:0016757, GO:0008152), and (GO:0016757, GO:0016021) have been calculated in the operation (f). Thus, the gene distance between the first candidate biomarker “B3GNTL1” and the second candidate biomarker “PLD5” may be derived by averaging the three GO term distances.

### GO Term Distances for Calculating Gene Distances

As described above, a GO term distance is a value showing the distance (in terms of function) between two GO terms. In some embodiments, the processor 13 calculates each of the GO term distances based on a corresponding information content distance and a corresponding Czekanowski-Dice distance (e.g., averaging the information content distance and the Czekanowski-Dice distance). Before calculating the information content distances and the Czekanowski-Dice distances, the processor 13 calculates a weight for each of the GO terms. The weight of a GO term can be considered as an indicator for the position of the GO term located in the GO tree.

For the i^{th} GO term, its weight is defined as the number of the candidate biomarkers CB_1, . . . , CB_k annotated by the i^{th} GO term divided by the number of non-duplicated candidate biomarkers CB_1, . . . , CB_k annotated by all the GO terms. A GO term located in an upper level of the GO tree correspond to more candidate biomarkers than a GO term located in lower level branches of the GO tree, and its corresponding weight would be relatively higher.

Two concrete examples are given herein with the assumption that 70 candidate biomarkers are annotated by the GO term “GO:0016757,” 690 candidate biomarkers are annotated by the GO term “GO:0003824,” and 20,987 non-duplicated candidate biomarkers are annotated by GO terms. Under the assumption, the weight of the GO term “GO:0016757” is 0.003335 approximately (i.e., 70/20,987≈0.003335), and the weight of the GO term “GO:0003824” is 0.032877 approximately (i.e., 690/20,987≈0.032877).

The information content distance between two GO terms is elaborated herein. If two GO terms belong to different biological concepts in the GO tree, the information content distance between them is defined as 1 (i.e., a value representing the farthest distance) because they do not have Lowest Common Ancestor (LCA). If two GO terms belong to the same biological concept in the GO tree, the two GO terms have one or more LCAs. If there is more than one LCA, the common ancestor with the lowest weight value is selected. For the case that two GO terms belong to the same biological concept in the GO tree, the information content distance between them is calculated based on the weights of the two GO terms as well as the weight of the LCA. The calculation of the information content distance of any two GO terms can be characterized by the following equation (5).

$${dist}_{IC} ( {t}_{i} , {t}_{j} ) = \{ \begin{matrix}2 W ( {t}_{{LCA}_{i , j}} ) - W ( {t}_{i} ) - W ( {t}_{j} ) , & {t}_{LCA} exist \\ 1 , & otherwise\end{matrix} \qquad ( 5 )$$

In the above equation (5), tᵢ represents the i^{th} GO term, tⱼ represents the j^{th} GO term, t_{LCAi,j} represents the LCA of the i^{th} and j^{th} GO terms, W(tᵢ) represents the weight of the i^{th} GO term, W(tⱼ) represents the weight of the j^{th} GO term, W (t_{LCAi,j}) represents weight of the GO term t_{LCAi,j}, and dist_{IC}(tᵢ, tⱼ) represents the information content distance between the i^{th} and j^{th} GO terms.

A concrete example regarding the information content distance is given herein. It is assumed that the GO term “GO:0016757” and the GO term “GO:0003824” has an LCA having the weight 0.036451. Under this assumption, the information content distance between the GO term “GO:0016757” and the GO term “GO:0003824” is 0.03669 (i.e., 2×0.036451−0.003335−0.032877=0.03669).

The Czekanowski-Dice distance between two GO terms is elaborated herein. The Czekanowski-Dice distance represents the similarity of the sets of the candidate biomarkers annotated by the two GO terms. It is assumed that Gₜ_{ᵢ} and Gₜ_{ⱼ} represent the sets of the candidate biomarkers annotated by the i^{th} and j^{th} GO terms respectively. The Czekanowski-Dice distance between the i^{th} and j^{th} GO terms can be calculated based on the following equation (6).

$${dist}_{CD} ( {t}_{i} , {t}_{j} ) = \frac{\vert {G}_{{t}_{i}} Δ {G}_{{t}_{j}} \vert}{\vert {G}_{{t}_{i}} ⋃ {G}_{{t}_{j}} \vert + \vert {G}_{{t}_{i}} ⋂ {G}_{{t}_{j}} \vert} \qquad ( 6 )$$

In the above equation (6), tᵢ represents the i^{th} GO term, tⱼ represents the j^{th} GO term, Gₜ_{ᵢ} represents the set of the candidate biomarkers annotated by the i^{th} GO term, Gₜ_{ⱼ} represents the set of the candidate biomarkers annotated by the j^{th} GO term, and dist_{CD}(tᵢ, tⱼ) represents the Czekanowski-Dice distance between the i^{th} and j^{th} GO terms. In addition, Gₜ_{ᵢ}ΔGₜ_{ⱼ} is the symmetrical difference between the sets Gₜ_{ᵢ} and Gₜ_{ⱼ}, Gₜ_{ᵢ}∪Gₜ_{ⱼ} is the union of the sets Gₜ_{ᵢ} and Gₜ_{ⱼ}, and Gₜ_{ᵢ}∩Gₜ_{ⱼ} is the intersection of the sets Gₜ_{ᵢ} and Gₜ_{ⱼ}. When the number of exclusive candidate biomarkers between the i^{th} and j^{th} GO terms is high, the Czekanowski-Dice distance between the i^{th} and j^{th} GO terms is relatively large.

A concrete example regarding the Czekanowski-Dice distance is given herein. Regarding the GO term “GO:0016757” and the GO term “GO:0003824,” it is assumed that the number of the exclusive candidate biomarkers is 694, the number of the union of the candidate biomarkers is 694, and the number of the intersection of the candidate biomarkers is 0. Under this assumption, the Czekanowski-Dice distance between the GO term “GO:0016757” and the GO term “GO:0003824” is 1.

### Algorithms for Biomarker Functional Clustering

As described above, in some embodiments, the processor 13 further clusters the candidate biomarkers CB_1, . . . , CB_k into the functional clusters G_1, . . . , G_p.

In some embodiments, the processor 13 adopts a partition clustering algorithm (e.g., K-means clustering method) to cluster the candidate biomarkers CB_1, . . . , CB_k into the functional clusters G_1, . . . , G_p based on the gene distances between every pair of the candidate biomarkers CB_1, . . . , CB_k.

Table 13 to Table 21 shows several examples of the clustering results by using the K-means clustering method. Specifically, Table 13 is for the target disease “colorectal cancer,” Table 14 is for the target disease “lung cancer,” Table 15 is for the target disease “liver cancer,” Table 16 is for the target disease “pancreatic cancer,” Table 17 is for the target disease “prostate cancer,” Table 18 is for the target disease “breast cancer,” Table 19 is for the target disease “ovarian cancer,” Table 20 is for the target disease “esophagus cancer,” and Table 21 is for the target disease “stomach cancer.” In these examples, the candidate biomarkers CB_1, . . . , CB_k being clustered are the intersection of the aforesaid exemplary primary biomarkers PB_1, . . . , PB_m and the aforesaid exemplary secondary biomarkers SB_1, . . . , SB_n.

TABLE 13
(K-means clustering result for the target disease “colorectal cancer”)

| K-means clustering | Number of candidate | Representative | Representative | Representative | Representative |
|---|---|---|---|---|---|
| group | biomarkers | KEGG^{#} pathway | GO Term BP | GO Term CC | GO Term MF |
| 1 | 30 | Herpes simplex | Regulation of | Nucleus | RNA polymerase II |
| | | virus 1 infection | transcription from | | transcription factor |
| | | | RNA polymerase II | | activity, sequence- |
| | | | promoter | | specific DNA binding |
| 2 | 65 | Yersinia infection | Positive regulation of | Chromatin | RNA polymerase II |
| | | | transcription from | | transcription factor |
| | | | RNA polymerase II | | activity, sequence- |
| | | | promoter | | specific DNA binding |
| 3 | 42 | Neuroactive ligand- | Potassium ion | Plasma membrane | Potassium channel |
| | | receptor interaction | transmembrane | | activity |
| | | | transport | | |

^{#}KEGG is the abbreviation of Kyoto Encyclopedia of Genes and Genomes

TABLE 14
(K-means clustering result for the target disease “lung cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 17 | Transcriptional | regulation of | chromatin | RNA polymerase II |
| | | misregulation in | transcription from | | transcription factor |
| | | cancer | RNA polymerase II | | activity, sequence- |
| | | | promoter | | specific DNA binding |
| 2 | 11 | none | none | glutamatergic | none |
| | | | | synapse | |
| 3 | 40 | Chemokine | Wnt signaling | Golgi membrane | protein self- |
| | | signaling pathway | pathway | | association |
| 4 | 8 | none | none | none | none |
| 5 | 52 | Signaling pathways | regulation of | chromatin | RNA polymerase II |
| | | regulating | transcription from | | transcription factor |
| | | pluripotency of | RNA polymerase II | | activity, sequence- |
| | | stem cells | promoter | | specific DNA binding |

TABLE 15
(K-means clustering result for the target disease “liver cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 57 | Pathways in cancer | positive regulation of | nucleus | sequence-specific |
| | | | transcription from | | DNA binding |
| | | | RNA polymerase II | | |
| | | | promoter | | |
| 2 | 18 | none | regulation of | integral component | calcium ion binding |
| | | | potassium ion | of membrane | |
| | | | transmembrane | | |
| | | | transport | | |
| 3 | 43 | Neuroactive ligand- | cell adhesion | plasma membrane | calcium ion binding |
| | | receptor interaction | | | |
| 4 | 58 | Calcium signaling | inflammatory | plasma membrane | protein binding |
| | | pathway | response | | |
| 5 | 30 | Hematopoietic cell | regulation of | proteinaceous | metal ion binding |
| | | lineage | transcription, DNA- | extracellular matrix | |
| | | | templated | | |
| 6 | 9 | none | peptidyl-serine | intracellular | zinc ion binding |
| | | | phosphorylation | | |

TABLE 16
(K-means clustering result for the target disease “pancreatic cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 28 | CAMP signaling | regulation of | chromatin | RNA polymerase II |
| | | pathway | transcription from | | transcription factor |
| | | | RNA polymerase II | | activity, sequence- |
| | | | promoter | | specific DNA binding |
| 2 | 9 | none | none | none | none |
| 3 | 49 | Insulin secretion | adherens junction | plasma membrane | protein kinase C |
| | | | organization | | binding |
| 4 | 18 | none | regulation of | chromatin | RNA polymerase II |
| | | | transcription from | | transcription factor |
| | | | RNA polymerase II | | activity, sequence- |
| | | | promoter | | specific DNA binding |
| 5 | 11 | none | none | none | none |
| 6 | 3 | none | none | none | none |
| 7 | 33 | Synaptic vesicle | neurotransmitter | plasma membrane | calcium ion binding |
| | | cycle | secretion | | |

TABLE 17
(K-means clustering result for the target disease “prostate cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 25 | Nicotinate and | oxidation-reduction | extracellular exosome | protein |
| | | nicotinamide | process | | homodimerization |
| | | metabolism | | | activity |
| 2 | 33 | Carbohydrate | extracellular matrix | plasma membrane | calcium ion binding |
| | | digestion and | organization | | |
| | | absorption | | | |
| 3 | 15 | none | none | extracellular exosome | none |
| 4 | 26 | Transcriptional | transcription from | cytosol | protein binding |
| | | misregulation in | RNA polymerase II | | |
| | | cancer | promoter | | |
| 5 | 31 | Pathways in cancer | positive regulation of | nucleus | DNA binding |
| | | | transcription from | | |
| | | | RNA polymerase II | | |
| | | | promoter | | |

TABLE 18
(K-means clustering result for the target disease “breast cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 15 | none | none | integral component | actin binding |
| | | | | of membrane | |
| 2 | 28 | Hematopoietic cell | antigen processing | proteinaceous | zinc ion binding |
| | | lineage | and presentation, | extracellular matrix | |
| | | | exogenous lipid | | |
| | | | antigen via MHC | | |
| | | | class Ib | | |
| 3 | 78 | Pathways in cancer | positive regulation of | nucleus | transcription factor |
| | | | transcription from | | activity, sequence- |
| | | | RNA polymerase II | | specific DNA binding |
| | | | promoter | | |
| 4 | 76 | Calcium signaling | transcription, DNA- | nucleus | transcription factor |
| | | pathway | templated | | activity, sequence- |
| | | | | | specific DNA binding |
| 5 | 45 | Cell adhesion | cell adhesion | plasma membrane | structural molecule |
| | | molecules (CAMs) | | | activity |

TABLE 19
(K-means clustering result for the target disease “ovarian cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 3 | none | none | none | none |
| 2 | 61 | Viral carcinogenesis | transcription, DNA- | nucleus | protein binding |
| | | | templated | | |
| 3 | 68 | none | negative regulation of | cytoplasm | identical protein |
| | | | neuron projection | | binding |
| | | | development | | |

TABLE 20
(K-means clustering result for the target disease “esophagus cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 31 | Neuroactive ligand- | cell adhesion | integral component | none |
| | | receptor interaction | | of membrane | |
| 2 | 29 | Basal cell | positive regulation of | nucleus | DNA binding |
| | | carcinoma | transcription from | | |
| | | | RNA polymerase II | | |
| | | | promoter | | |
| 3 | 19 | none | transcription, DNA- | nucleus | DNA binding |
| | | | templated | | |
| 4 | 23 | Transcriptional | transcription, DNA- | nucleus | sequence-specific |
| | | misregulation in | templated | | DNA binding |
| | | cancer | | | |
| 5 | 48 | Neuroactive ligand- | positive regulation of | plasma membrane | receptor binding |
| | | receptor interaction | GTPase activity | | |

TABLE 21
(K-means clustering result for the target disease “stomach cancer”)

| K-means clustering | Number of candidate | Representative | Representative | Representative | Representative |
|---|---|---|---|---|---|
| group | biomarkers | KEGG pathway | GO Term BP | GO Term CC | GO Term MF |
| 1 | 36 | Ras signaling | innate immune | intracellular | metal ion binding |
| | | pathway | response | | |
| 2 | 68 | MicroRNAs in | positive regulation of | cytoplasm | protein binding |
| | | cancer | GTPase activity | | |
| 3 | 44 | Pathways in cancer | negative regulation of | nucleus | transcription factor |
| | | | transcription from | | activity, sequence- |
| | | | RNA polymerase II | | specific DNA binding |
| | | | promoter | | |
| 4 | 36 | none | cell adhesion | plasma membrane | actin filament |
| | | | | | binding |
| 5 | 27 | none | transcription, DNA- | nucleus | protein binding |
| | | | templated | | |
| 6 | 20 | none | membrane raft | integral component | structural constituent |
| | | | polarization | of membrane | of myelin sheath |

In some embodiments, the processor 13 adopts a hierarchical clustering algorithm (e.g., the unweighted pair-group method with arithmetic mean (UPGMA)) to cluster the candidate biomarkers CB_1, . . . , CB_k into the functional clusters G_1, . . . , G_p based on the gene distances between every pair of the candidate biomarkers CB_1, . . . , CB_k.

Table 22 shows several examples of the clustering results by using the UPGMA method. In these examples, the candidate biomarkers CB_1, . . . , CB_k being clustered are the intersection of the aforesaid exemplary primary biomarkers PB_1, . . . , PB_m and the aforesaid exemplary secondary biomarkers SB_1, . . . , SB_n.

TABLE 22
(UPGMA clustering results for nine target diseases)

| | | Number of candidate |
|---|---|---|
| Target disease | UPGMA clustering group | biomarkers |
| Colorectal cancer | 1 | 77 |
| | 2 | 28 |
| | 3 | 31 |
| Lung cancer | 1 | 24 |
| | 2 | 104 |
| Liver cancer | 1 | 106 |
| | 2 | 109 |
| Pancreatic cancer | 1 | 94 |
| | 2 | 54 |
| | 3 | 3 |
| Prostate cancer | 1 | 80 |
| | 2 | 20 |
| | 3 | 29 |
| Breast cancer | 1 | 166 |
| | 2 | 73 |
| Ovarian cancer | 1 | 106 |
| | 2 | 23 |
| | 3 | 3 |
| Esophagus cancer | 1 | 37 |
| | 2 | 112 |
| Stomach cancer | 1 | 170 |
| | 2 | 58 |

Weight calculation and target biomarker selection

As described above, different candidate biomarkers CB_1, . . . , CB_k represent different functional roles, and candidate biomarkers within the same functional cluster are close to each other in terms of function. Therefore, to understanding the relation between the target disease and at least one category of function(s), at least one of the functional clusters G_1, . . . , G_p may be further investigated.

In some embodiments, all the functional clusters G_1, . . . , G_p are further investigated. The processor 13 calculates a weight for each of the candidate biomarkers in each of the functional clusters G_1, . . . , G_p. The weight of a candidate biomarker indicates its importance within the functional cluster that it belongs to. Within a functional cluster, the higher the weight is, the more representative the corresponding candidate biomarker is for that functional cluster.

In some embodiments, the processor 13 determines at least one target biomarker from at least one of the functional clusters according to the weights in each of the functional clusters G_1, . . . , G_p. As shown in the example in FIG. 4, the processor 13 determines two target biomarkers Ta, Tb from the functional cluster G_1 according to the weights of the candidate biomarkers in the functional cluster G_1 but determines none target biomarker from the functional clusters G_p according to the weights of the candidate biomarkers in the functional cluster G_p.

The processor 13 can determine at least one target biomarker from at least one of the functional clusters according to the weights in each of the functional clusters G_1, . . . , G_p based on different strategies. In some embodiments, given a functional cluster, the processor 13 may select the candidate biomarker(s) whose weight is/are greater than a third predetermined threshold as the target biomarker(s). In some embodiments, the processor 13 can rank the candidate biomarkers in each of the functional clusters G_1, . . . , G_p according to the corresponding weights. For those embodiments, the processor 13 can determine the target biomarker(s) for each of the functional clusters G_1, . . . , G_p according to the corresponding ranking result.

The above description regarding weight calculation and target biomarker selection is for the case that all the functional clusters G_1, . . . , G_p are further investigated. As mentioned, it is also feasible that only one or some of the functional clusters G_1, . . . , G_p are further investigated. A person having ordinary skill in the art shall understand how to modify the aforesaid operations for the case that only one or some of the functional clusters G_1, . . . , G_p are further investigated and, thus, the details are not described herein.

### Recurrent Neural Network for Weight Calculation

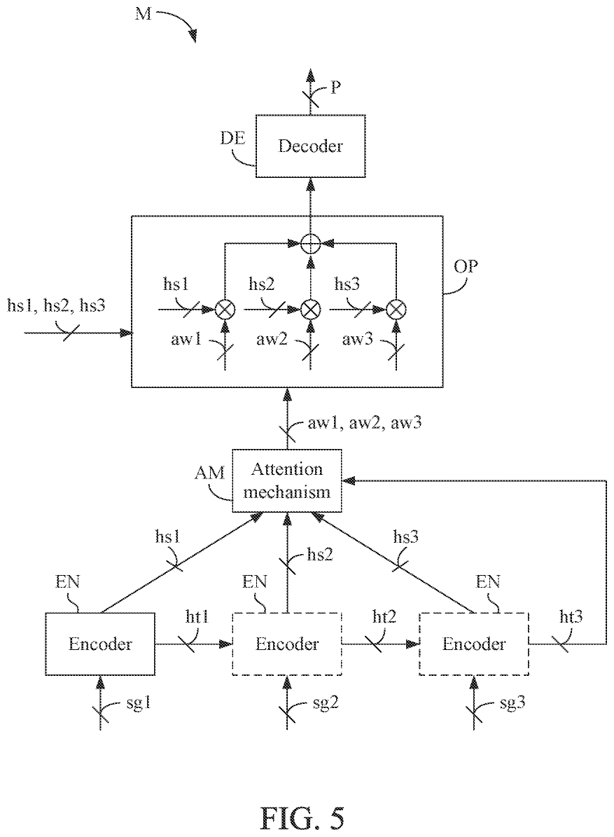

In some embodiments, the processor 13 executes a recurrent neural network M and calculates the weight of each of the candidate biomarkers in each of the functional clusters G_1, . . . , G_p by the recurrent neural network M. As shown in FIG. 5, the recurrent neural network M is attention-based and comprises an encoder EN, an attention mechanism AM, and a decoder DE, wherein the attention mechanism AM may be a two-layer fully connected network. Please note that there is only one encoder EN in the recurrent neural network M. Although more than one encoder EN is shown in FIG. 5, they are shown to represent that the encoder EN executes several times (will be elaborated later). The recurrent neural network M can be trained for outputting a prediction P regarding whether an inputted biomarker sequence corresponds to a subject having the target disease (will be elaborated later).

In those embodiments, the storage 11 stores a plurality of candidate biomarker sequences D3_1, . . . , D3_s, which may be retrieved from a fifth database through a transceiving interface (not shown) of the methylation biomarker selection apparatus 1. Each of the candidate biomarker sequences D3_1, . . . , D3_s corresponds to one of the candidate biomarkers CB_1, . . . , CB_k. The candidate biomarker sequences D3_1, . . . , D3_s are classified into a normal subject group or a disease subject group. The normal subject group comprises the candidate biomarker sequences related to the subjects without the target disease, while the disease subject group comprises the candidate biomarker sequences related to the subjects with the target disease.

In those embodiments, the processor 13 calculates the weight for each of the candidate biomarkers in each of the functional clusters G_1, . . . , G_p by the following operations (h), (i), (j), (k), and (l).

In the operation (h), the processor 13 derives a plurality of normal attention weights from the attention mechanism AM by inputting the candidate biomarker sequences corresponding to the candidate biomarker and from the normal subject group into the recurrent neural network M.

A concrete example is given herein for better understanding. It is assumed that the processor 13 is handling the functional cluster G_p, and the functional cluster G_p comprises three candidate biomarker gp1, gp2, gp3. It is also assumed that the candidate biomarker sequences comprised in the normal subject group correspond to N normal subjects (i.e., N subjects without the target disease), wherein N is a positive integer. For each of the N normal subjects, his or her candidate biomarker sequence sg1, sg2, sg3 respectively corresponding to the candidate biomarker gp1, gp2, gp3 are inputted to the encoder EN in sequence. As shown in FIG. 5, the encoder EN outputs a feedback vector ht1 and a status vector hs1 in response to the candidate biomarker sequence sg1, outputs a feedback vector ht2 and a status vector hs2 in response to the candidate biomarker sequence sg2 and the feedback vector ht1, and outputs a feedback vector ht3 and a status vector hs3 in response to the candidate biomarker sequence sg3 and the feedback vector ht2. The attention mechanism AM outputs the normal attention weight aw1, aw2, aw3 in response to the status vectors hs1, hs2, hs3 and the feedback vector ht3, wherein the normal attention weight aw1, aw2, aw3 respectively correspond to the candidate biomarker gp1, gp2, gp3. After the candidate biomarker sequences of all the N normal subjects have been processed, N normal attention weights for each of the candidate biomarker gp1, gp2, gp3 will be derived.

Although the above concrete example is for the functional cluster G_p, a person having ordinary skill in the art shall understand that the normal attention weights corresponding to the candidate biomarker(s) in each of the rest functional clusters can be derived by the same approach. Hence, the details are not repeated.

In the operation (i), the processor 13 derives a plurality of disease attention weights from the attention mechanism AM by inputting the candidate biomarker sequences corresponding to the candidate biomarker and from the disease subject group into the recurrent neural network. The operation (i) is similar to the operation (h), and the only difference is that the operation (i) is applied to candidate biomarker sequences from the disease subject group. A person having ordinary skill in the art shall understand the details of the operation (i) based on the above description of the operation (h).

In the operation (j), the processor 13 calculates an averaged normal weight by averaging the normal attention weights. Taking the candidate biomarker gp1 as an example, the processor 13 calculates the averaged normal weight corresponding to the candidate biomarker gp1 by averaging the normal attention weights corresponding to the candidate biomarker gp1. Please note that the processor 13 calculates an averaged normal weight for each of the candidate biomarkers in each of the functional clusters G_1, . . . , G_p.

In the operation (k), the processor 13 calculates an averaged disease weight by averaging the disease attention weights. Similarly, taking the candidate biomarker gp1 as an example, the processor 13 calculates the averaged disease weight corresponding to the candidate biomarker gp1 by averaging the disease attention weights corresponding to the candidate biomarker gp1. Please also note that the processor 13 calculates an averaged disease weight for each of the candidate biomarkers in each of the functional clusters G_1 . . . G_p.

In the operation (1), the processor 13 calculates the weight according to the averaged normal weight and the averaged disease weight. Again, taking the candidate biomarker gp1 as an example, the processor 13 calculates the weight of the candidate biomarker gp1 according to the averaged normal weight of the candidate biomarker gp1 and the averaged disease weight of the candidate biomarker gp1. Similarly, the processor 13 calculates the weight for each of the candidate biomarkers in each of the functional clusters G_1, . . . , G_p.

The advantage of using the recurrent neural network M for weight calculation is that the recurrent neural network M is good at handling long data sequence. Adopting a conventional neural network model usually has the technical problem of lacking sufficient space for storing long data sequence. The attention mechanism AM of the recurrent neural network M has the ability to ignore less important data. As only more important data is stored, adopting the recurrent neural network M for weight calculation will not face the technical problem of lacking sufficient space for storing data.

As described above, the recurrent neural network M can be trained for outputting a prediction P regarding whether the inputted biomarker sequences correspond to a subject having the target disease. In the example (i.e., the example that the inputted biomarker sequences are the candidate biomarker sequence sg1, sg2, sg3) shown in FIG. 5, the weighted summation operation OP generates a signal by weighting the status vectors hs1, hs2, hs3 by the normal attention weight aw1, aw2, aw3 respectively and then sums them up, and then the decoder DE generates the prediction P in response to the signal from the weighted summation operation OP.

### Candidate Biomarker Validation

In some embodiments, to achieve more accurate result, the processor 13 validates the candidate biomarkers CB_1, . . . , CB_k before performing biomarker functional clustering and eliminates the candidate biomarker(s) that fail(s) the validation. Candidate biomarker validation comprises two stages, including optimal cut-point selection and candidate biomarker screening.

In the first stage, the processor 13 determines an optimal cut-point from a plurality of preset cut-points for each of the candidate biomarkers CB_1, . . . , CB_k by the following operations (m), (n), (o), and (p). The optimal cut-point of a candidate biomarker may be considered as a threshold for determining whether a methylation degree corresponding to this candidate biomarker is severe. A preset cut-point may be a value between 0 and the maximum value of the methylation degree. It is noted that the present invention does not limit the number of the preset cut-points. Nevertheless, more preset cut-points will result in more accurate optimal cut-point. As an example, if the maximum value of the methylation degree is 1 and 99 preset cut-points are desired, the values of the 99 preset cut-points can be set to 0.01, 0.02, . . . , and 0.99.

In the operation (m), the processor 13 calculates an averaged normal value according to the methylation degrees corresponding to the concerned candidate biomarker (e.g., the candidate biomarkers CB_1) from the normal subject group based on the first data sets D1_1, . . . , D1_q. Please note that if the averaged normal value has been calculated (e.g., the aforesaid operation (a) has been executed), the operation (m) can be omitted.

In the operation (n), the processor 13 calculates a plurality of first difference values by subtracting the averaged normal value from each of the methylation degrees corresponding to the concerned candidate biomarker (e.g., the candidate biomarkers CB_1) recorded in the first data sets D1_1, . . . , D1_q.

In the operation (o), the processor 13 generates a first confusion matrix for each of the preset cut-points according to the first difference values corresponding to the concerned candidate biomarker (e.g., the candidate biomarkers CB_1).

A concrete example is given herein for better understanding. The first confusion matrix for a concerned candidate biomarker (e.g., the candidate biomarkers CB_1) and a concerned preset cut-point (e.g., 0.02) comprises the following four statistical numbers: (i) the total number of the subjects that are predicted as having the target disease and do have the target disease, which is represented by the variable N_{TP}, (ii) the total number of the subjects that are predicted as having the target disease but do not have the target disease, which is represented by the variable N_{FP}, (iii) the total number of the subjects that are predicted as not having the target disease but do have the target disease, which is represented by the variable N_{FN}, and (iv) the total number of the subjects that are predicted as not having the target disease and actually not have the target disease, which is represented by the variable N_{TN}.

For a first difference value, if it is greater than the concerned preset cut-point (e.g., 0.02), it is predicted that the corresponding subject has the target disease. In addition, whether a subject corresponding to a first difference value has the target disease is known because a first difference value is calculated based on a methylation degree recorded in one of the first data sets D1_1, . . . , D1_q, and each of the first data sets D1_1, . . . , D1_q belongs to the normal subject group or the target subject group.

In the operation (p), the processor 13 selects one of the preset cut-points as the optimal cut-point for the concerned candidate biomarker (e.g., the candidate biomarkers CB_1) according to the corresponding first confusion matrixes.

For a concerned candidate biomarker (e.g., the candidate biomarkers CB_1), a first confusion matrix for each of the preset cut-points has been generated in the operation (o). For example, if there are 99 preset cut-points, there will be 99 first confusion matrixes correspond to the concerned candidate biomarkers. In some embodiments, for each of the first confusion matrixes, the processor 13 can generate a sensitivity value (i.e., N_{TP}/(N_{TP}+N_{FN})) and a specificity value (i.e., N_{TN}/(N_{TN}+N_{FP})) based on the first confusion matrix and then generates a summarized value of the sensitivity value and the specificity value. Then, the processor 13 selects the preset cut-point with the greatest summarized value as the optimal cut-point for the concerned candidate biomarker.

The second stage (i.e., candidate biomarker screening) is described herein. To perform the second stage, the storage 11 stores a plurality of third data sets D4_1, . . . , D4_t, each of the third data sets D4_1, . . . , D4_t comprises a plurality of methylation degrees corresponding to the methylation loci. The methylation biomarker selection apparatus 1 may derives the third data sets D4_1, . . . , D4_t from a sixth database (e.g., Gene Expression Omnibus (GEO) database) through a transceiving interface (not shown) of the methylation biomarker selection apparatus 1.

Examples regarding the information related to the third data sets D4_1, . . . , D4_t used for nine target diseases are shown in Table 23. Please note that the data files from TCGA are of Mar. 15, 2021, and the data files from GEO database are of Oct. 30, 2021. In addition, the variable N_{N} represents the number of the subject without the target disease, and the variable N_{TD} represents the number of the subject with the target disease.

TABLE 23

| | Sixth | |
|---|---|---|
| Target disease | database | N_{N}/N_{TD} |
| Colorectal cancer | GEO | 0/48 |
| Lung cancer | GEO | 19/164 |
| Liver cancer | GEO | 22/22 |
| Pancreatic cancer | GEO | 29/167 |
| Prostate cancer | GEO | 16/31 |
| Breast cancer | TCGA | 47/368 |
| Ovarian cancer | GEO | 10/10 |
| Esophagus cancer | GEO | 16/24 |
| Stomach cancer | GEO | 12/12 |

The processor 13 validates each of the candidate biomarkers CB_1, . . . , CB_k by the following operations (q), (r), (s), and (t).

In the operation (q), the processor 13 calculates a plurality of second difference values by subtracting the averaged normal value from each of the methylation degrees corresponding to the candidate biomarker and from the third data sets D4_1, . . . , D4_t.

In the operation (r), the processor 13 generates a second confusion matrix for the optimal cut-point according to the optimal cut-point and the second difference values corresponding to the candidate biomarker. Similarly, the second confusion matrix comprises the following four statistical numbers: (i) the total number of the subjects that are predicted as having the target disease and do have the target disease, (ii) the total number of the subjects that are predicted as having the target disease but do not have the target disease, (iii) the total number of the subjects that are predicted as not having the target disease but do have the target disease, and (iv) the total number of the subjects that are predicted as not having the target disease and actually not have the target disease.

In the operation (s), the processor 13 generates a sensitivity value, a specificity value, and an accuracy value (i.e., the ratio that the prediction is correct) according to the second confusion matrix. For better understanding, please refer to Table 24 for the statistics of the accuracy values of the candidate biomarkers of each of the nine target diseases.

TABLE 24

| | Number of candidate | Top 10 classification | Top 20 classification | Total classification |
|---|---|---|---|---|
| Target disease | biomarkers | accuracy average | accuracy average | accuracy average |
| Colorectal cancer | 141 | 0.933333 | 0.913542 | 0.8125 |
| Lung cancer | 135 | 0.933333 | 0.922677 | 0.759191 |
| Liver cancer | 222 | 0.659091 | 0.631818 | 0.539312 |
| Pancreatic cancer | 156 | 0.960204 | 0.952296 | 0.85397 |
| Prostate cancer | 131 | 0.993617 | 0.98617 | 0.907001 |
| Breast cancer | 246 | 0.934934 | 0.921928 | 0.836189 |
| Ovarian cancer | 135 | 0.97 | 0.955 | 0.739474 |
| Esophagus cancer | 157 | 0.95 | 0.9225 | 0.707643 |
| Stomach cancer | 234 | 0.795833 | 0.76875 | 0.583511 |

In the operation (t), the processor 13 validates the candidate biomarker according to the accuracy value and a fourth predetermined threshold. For example, if the accuracy value of a candidate biomarker is lower than the fourth predetermined threshold, that candidate biomarker is eliminated.

For the embodiments that perform candidate biomarker validation, only candidate biomarkers that pass the validation (i.e., have not been eliminated) will be functional clustered.

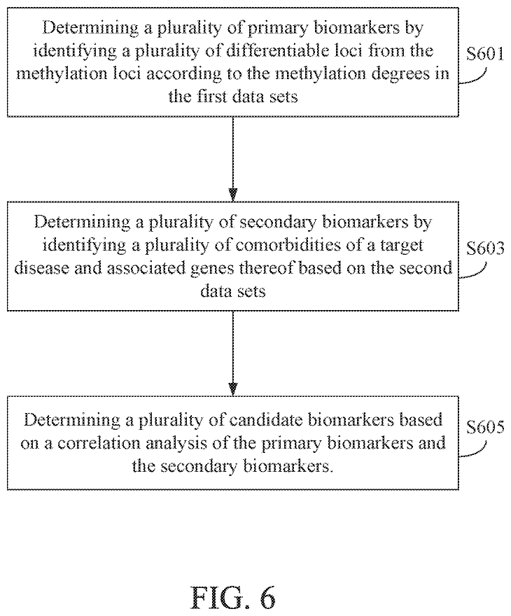

FIG. 6 illustrates the main flowchart of a methylation biomarker selection method in some embodiments of the present invention. The methylation biomarker selection method is for use in an electronic apparatus (e.g., the methylation biomarker selection apparatus 1). The electronic apparatus stores a plurality of first data sets and a plurality of second data sets, wherein each of the first data sets comprises a plurality of methylation degrees corresponding to a plurality of methylation loci and each of the second data sets comprises at least one medical record. The methylation biomarker selection method comprises the following steps S601, S603, and S605.

In the step S601, the electronic apparatus determines a plurality of primary biomarkers by identifying a plurality of differentiable loci from the methylation loci according to the methylation degrees in the first data sets. In some embodiments, the step S601 comprises a step of selecting the methylation loci having at least one of an averaged methylation degree difference conforming to a first predetermined rule and a p-value conforming to a second predetermined rule as the differentiable loci, wherein the differentiable loci are determined as the primary biomarkers.

In the step S603, the electronic apparatus determines a plurality of secondary biomarkers by identifying a plurality of comorbidities of a target disease, and associated genes thereof based on the second data sets. In some embodiments, the step S603 comprises a step of calculating an association degree indicating relevance to the target disease for each of the distinct diagnosed diseases, a step of selecting the diagnosed diseases having the association degree conforming to a third predetermined rule as the comorbidities, and a step of determining a plurality of genes corresponding to the comorbidities as the secondary biomarkers. In some embodiments, the association degree of each of the distinct diagnosed diseases comprises an odds ratio, a p-value, and a supporting rate.

In the step S605, the electronic apparatus determines a plurality of candidate biomarkers based on a correlation analysis of the primary biomarkers and the secondary biomarkers. Please note that the order for executing steps S601 and S603 is not limited by the present invention. In one example, the step S603 may be executed prior to the step S601. In another example, the step S601 and the step S603 may be executed at the same time.

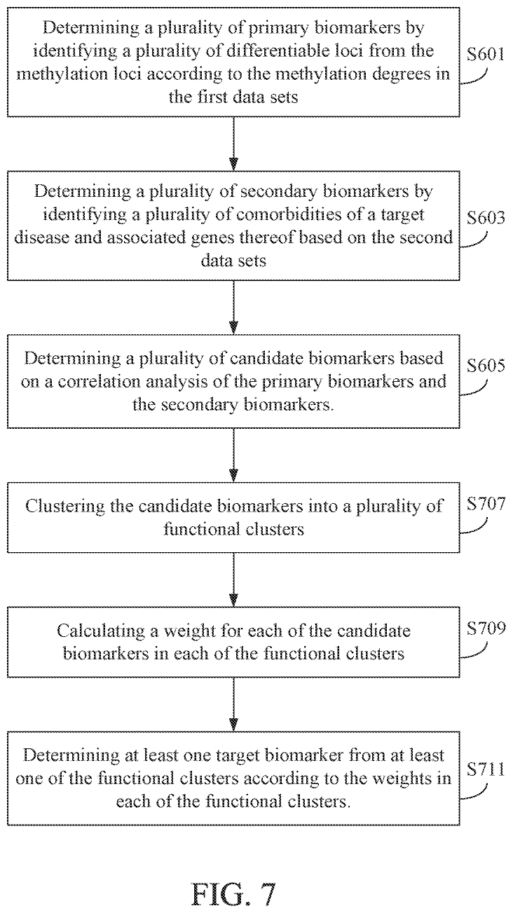

FIG. 7 illustrates the main flowchart of a methylation biomarker selection method in some embodiments of the present invention. In those embodiments, the methylation biomarker selection method further comprises the following steps S707, S709, and S711 in addition to the steps S601, S603, and S605.

In the step S707, the electronic apparatus clusters the candidate biomarkers into a plurality of functional clusters. In some embodiments, the step S707 clusters the candidate biomarkers into the functional clusters based on a plurality of gene distances between every pair of the candidate biomarkers. In those embodiments, the step S707 comprises a step of calculating at least one gene distance, which further comprises a step of calculating a GO term distance for each of at least one GO term pair between a first candidate biomarker and a second candidate biomarker and a step of determining the gene distance between the first candidate biomarker and the second candidate biomarker according to the at least one GO term distance. In some embodiments, each of the GO term distances is calculated based on an information content distance and a Czekanowski-Dice distance.

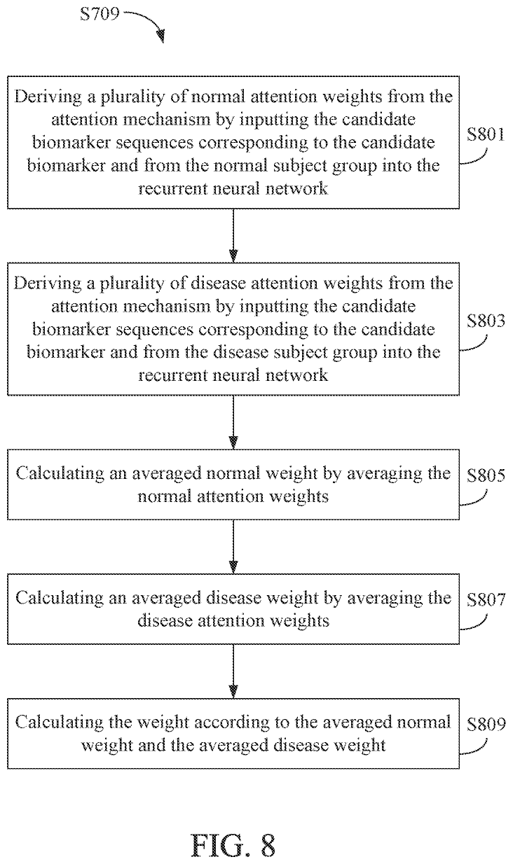

In the step S709, the electronic apparatus calculates a weight for each of the candidate biomarkers in each of the functional clusters. In some embodiments, the electronic apparatus executes a recurrent neural network comprising an encoder, an attention mechanism, and a decoder, and the step S709 is realized by a recurrent neural network. In those embodiments, each of a plurality of candidate biomarker sequences belongs to one of a normal subject group and a disease subject group, each of the candidate biomarker sequences corresponds to one of the candidate biomarkers, and the step S709 comprises the steps S801, S803, S805, S807, and S809 as shown in FIG. 8.

In the step S801, the electronic apparatus derives a plurality of normal attention weights from the attention mechanism by inputting the candidate biomarker sequences corresponding to the candidate biomarker and from the normal subject group into the recurrent neural network. In the step S803, the electronic apparatus derives a plurality of disease attention weights from the attention mechanism by inputting the candidate biomarker sequences corresponding to the candidate biomarker and from the disease subject group into the recurrent neural network. In the step S805, the electronic apparatus calculates an averaged normal weight by averaging the normal attention weights. In the step S807, the electronic apparatus calculates an averaged disease weight by averaging the disease attention weights. In the step S809, the electronic apparatus calculates the weight according to the averaged normal weight and the averaged disease weight. Please note that the steps S801, S803, S805, and S807 may be executed in other order as long as the step S801 is prior to the step S803 and the step S805 is prior to the step S807.

In the step S711, the electronic apparatus determines at least one target biomarker from at least one of the functional clusters according to the weights in each of the functional clusters. In some embodiments, the methylation biomarker selection method further comprises a step of ranking the candidate biomarkers in each of the functional clusters according to the corresponding weights. In those embodiments, the step S711 may determine the at least one target biomarker from at least one of the functional clusters according to the ranking result of each of the functional clusters.

In addition to the previously mentioned steps, the methylation biomarker selection method provided by the present invention can also execute all the operations and steps that can be executed by the methylation biomarker selection apparatus 1, have the same functions as the methylation biomarker selection apparatus 1, and deliver the same technical effects as the methylation biomarker selection apparatus 1. How the methylation biomarker selection method provided by the present invention executes these operations and steps, has the same functions, and delivers the same technical effects as the methylation biomarker selection apparatus 1 will be readily appreciated by a person having ordinary skill in the art based on the above explanation of the methylation biomarker selection apparatus 1 and, thus, will not be further described herein.

The methylation biomarker selection method described in the above embodiments may be implemented as a computer program comprising a plurality of codes. The computer program is stored in a non-transitory computer readable storage medium. After the codes of the computer program are loaded into an electronic apparatus (e.g., the methylation biomarker selection apparatus 1), the computer program executes the methylation biomarker selection method as described in the above embodiments. The non-transitory computer readable storage medium may be an electronic product, such as a Read Only Memory (ROM), a flash memory, a floppy disk, a hard disk, a Compact Disk (CD), a Digital Versatile Disc (DVD), a mobile disk, a database accessible to networks, or any other storage media with the same function and well-known to a person having ordinary skill in the art.

### Clinical Validation of Target Biomarkers for Colorectal Cancer

In order to confirm the utility of the candidate biomarkers in the clinical setting, the methylation-specific Polymerase Chain Reaction (PCR) strategy is utilized to accomplish the clinical validation on these candidate biomarkers of the colorectal cancer using DNA extracted from formalin-fixed, paraffin-embedded (FFPE) tumor tissue specimens. Taking colorectal cancer as an example, 10 target biomarkers are selected from 141 candidate biomarkers and designed the corresponding quantitative methylation-specific PCR (qMSP) primers for each target biomarker. First, the commercial human methylated and non-methylated DNA standards (Zymo research, Cat. #D5014) are used to test the primer performance and to build up the calibration curves for subsequent estimation of methylation levels in the clinical samples.

Next, 99 clinical FFPE samples are selected, including 18 normal tissues and 81 tumor tissues across 9 cancer types, to ascertain the methylation levels of these selected 10 target biomarkers of the colorectal cancer in various cancer specimens. The extracted DNA were underwent bisulfite conversion by using EZ DNA Methylation-Lightning™ kit (Zymo research, Cat. #D5031) following the manufacturer's instruction manual. Finally, the bisulfite-converted DNA were subjected to qMSP tests for further determining their methylation levels by using the calibration curves.

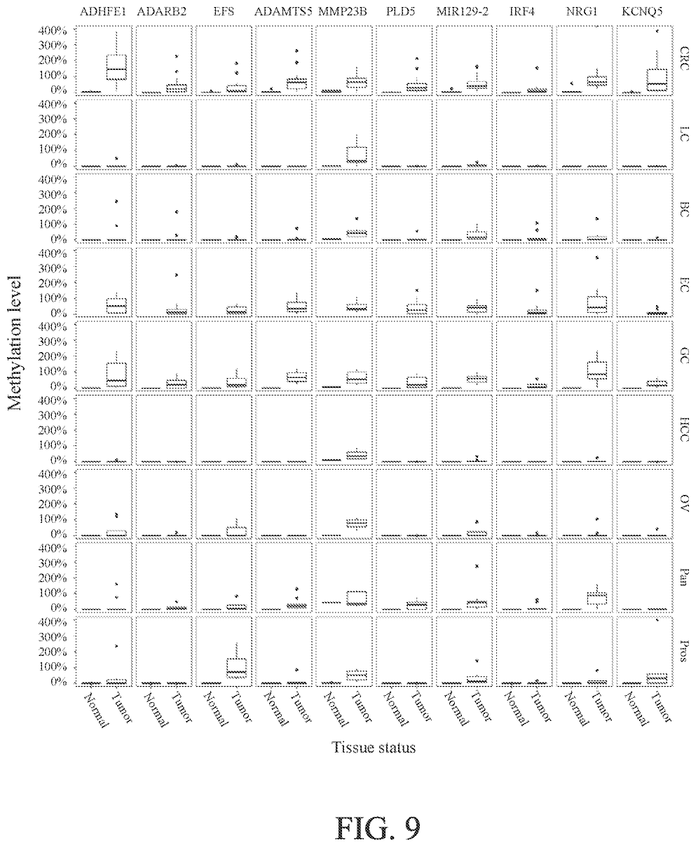

All the results are presented in FIG. 9 and Table 25 to Table 33 below. In FIG. 9, “CRC” stands for colorectal cancer, “LC” stands for lung cancer, “BC” stands for breast cancer, “EC” stands for esophageal cancer, “GC” stands for gastric cancer, “HCC” stands for hepatocellular carcinoma, “OV” stands for ovarian cancer, “Pan” stands for pancreatic cancer, and “Pros” stands for prostate cancer. In addition, Table 25 is for “colorectal cancer,” Table 26 is for “lung cancer,” Table 27 is for “breast cancer,” Table 28 is for “esophageal cancer,” Table 29 is for “gastric cancer,” Table 30 is for “hepatocellular carcinoma,” Table 31 is for “ovarian cancer,” Table 32 is for “pancreatic cancer,” and Table 33 is for “prostate cancer.”

The results reveal that the methylation levels of the target biomarkers of the colorectal cancer are significantly up-regulated in colorectal cancer tumor tissue compared to normal tissues. In addition, ADHFE1, PLD5, and NRGT had a higher methylation level in gastric (GC), esophageal (EC), and pancreatic (Pan) cancers. In contrast, the methylation extent of the MMP23B gene seemed to be elevated in every tested cancer type.

TABLE 25
(Clinical validation result for colorectal cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 5 | max | 14.4% | 0.9% | 7.3% | 21.1% | 15.8% | 6.5% | 22.6% | 0.0% | 56.2% | 3.1% |
| | | Q3 | 8.2% | 0.4% | 2.1% | 6.4% | 12.0% | 3.8% | 6.3% | 0.0% | 7.8% | 0.0% |
| | | median | 3.2% | 0.4% | 0.9% | 3.8% | 4.0% | 1.0% | 3.6% | 0.0% | 3.2% | 0.0% |
| | | Q1 | 0.9% | 0.1% | 0.7% | 2.4% | 1.8% | 0.4% | 0.4% | 0.0% | 2.7% | 0.0% |
| | | min | 0.7% | 0.0% | 0.0% | 1.8% | 1.7% | 0.0% | 0.1% | 0.0% | 1.6% | 0.0% |
| Tumor | 15 | max | 476.1% | 228.9% | 183.1% | 264.0% | 163.4% | 214.0% | 163.3% | 153.7% | 421.9% | 652.2% |
| | | Q3 | 234.8% | 48.1% | 44.0% | 83.7% | 88.0% | 54.5% | 70.4% | 20.9% | 98.0% | 146.7% |
| | | median | 147.7% | 21.8% | 9.4% | 62.8% | 66.2% | 29.6% | 42.5% | 9.3% | 65.9% | 53.2% |
| | | Q1 | 83.0% | 5.2% | 5.9% | 21.9% | 33.2% | 12.4% | 25.8% | 2.2% | 46.6% | 12.3% |
| | | min | 10.5% | 0.0% | 0.0% | 8.0% | 1.0% | 0.0% | 1.8% | 0.0% | 19.6% | 0.0% |

TABLE 26
(Clinical validation result for lung cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 2 | max | 0.0% | 0.3% | 0.0% | 0.0% | 5.8% | 0.0% | 0.0% | 0.0% | 0.0% | 0.0% |
| | | Q3 | 0.0% | 0.2% | 0.0% | 0.0% | 4.5% | 0.0% | 0.0% | 0.0% | 0.0% | 0.0% |
| | | median | 0.0% | 0.1% | 0.0% | 0.0% | 3.2% | 0.0% | 0.0% | 0.0% | 0.0% | 0.0% |
| | | Q1 | 0.0% | 0.1% | 0.0% | 0.0% | 2.0% | 0.0% | 0.0% | 0.0% | 0.0% | 0.0% |
| | | min | 0.0% | 0.0% | 0.0% | 0.0% | 0.7% | 0.0% | 0.0% | 0.0% | 0.0% | 0.0% |
| Tumor | 7 | max | 50.2% | 5.9% | 10.8% | 2.5% | 197.4% | 1.4% | 25.9% | 3.1% | 1.7% | 0.9% |
| | | Q3 | 1.6% | 0.4% | 2.5% | 1.8% | 122.2% | 0.6% | 11.4% | 0.0% | 1.3% | 0.0% |
| | | median | 0.9% | 0.0% | 0.8% | 0.4% | 33.3% | 0.4% | 3.2% | 0.0% | 0.9% | 0.0% |
| | | Q1 | 0.4% | 0.0% | 0.3% | 0.1% | 24.1% | 0.1% | 1.8% | 0.0% | 0.3% | 0.0% |
| | | min | 0.0% | 0.0% | 0.0% | 0.0% | 1.6% | 0.0% | 0.3% | 0.0% | 0.0% | 0.0% |

TABLE 27
(Clinical validation result for breast cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 1 | max | 0.0% | 0.0% | 0.2% | 0.1% | 4.7% | 0.8% | 0.3% | 0.0% | 0.0% | 0.4% |
| | | Q3 | 0.0% | 0.0% | 0.2% | 0.1% | 4.7% | 0.8% | 0.3% | 0.0% | 0.0% | 0.4% |
| | | median | 0.0% | 0.0% | 0.2% | 0.1% | 4.7% | 0.8% | 0.3% | 0.0% | 0.0% | 0.4% |
| | | Q1 | 0.0% | 0.0% | 0.2% | 0.1% | 4.7% | 0.8% | 0.3% | 0.0% | 0.0% | 0.4% |
| | | min | 0.0% | 0.0% | 0.2% | 0.1% | 4.7% | 0.8% | 0.3% | 0.0% | 0.0% | 0.4% |
| Tumor | 9 | max | 244.8% | 177.2% | 21.4% | 72.4% | 135.3% | 56.2% | 105.3% | 107.1% | 135.6% | 16.1% |
| | | Q3 | 1.2% | 1.4% | 0.5% | 3.4% | 59.2% | 3.3% | 51.6% | 8.2% | 23.3% | 0.8% |
| | | median | 0.5% | 0.4% | 0.3% | 1.0% | 42.9% | 1.2% | 19.0% | 0.5% | 2.1% | 0.3% |
| | | Q1 | 0.3% | 0.0% | 0.1% | 0.7% | 23.5% | 0.0% | 5.6% | 0.0% | 0.5% | 0.0% |
| | | min | 0.0% | 0.0% | 0.0% | 0.0% | 16.6% | 0.0% | 0.4% | 0.0% | 0.0% | 0.0% |

TABLE 28
(Clinical validation result for esophageal cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Tumor | 10 | max | 135.8% | 245.6% | 65.6% | 135.0% | 105.7% | 149.0% | 96.8% | 148.3% | 356.7% | 46.3% |
| | | Q3 | 98.7% | 31.1% | 44.7% | 73.3% | 58.9% | 59.1% | 51.7% | 24.5% | 108.8% | 8.7% |
| | | median | 50.1% | 10.4% | 11.3% | 34.8% | 33.6% | 25.4% | 39.3% | 7.5% | 40.5% | 4.3% |
| | | Q1 | 6.1% | 0.7% | 2.7% | 13.2% | 25.0% | 2.1% | 11.7% | 0.6% | 9.3% | 0.0% |
| | | min | 0.0% | 0.0% | 0.1% | 0.0% | 6.7% | 0.0% | 0.1% | 0.0% | 0.0% | 0.0% |

TABLE 29
(Clinical validation result for gastric cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 1 | max | 0.3% | 0.0% | 0.1% | 0.2% | 6.1% | 0.0% | 0.1% | 0.0% | 0.1% | 0.0% |
| | | Q3 | 0.3% | 0.0% | 0.1% | 0.2% | 6.1% | 0.0% | 0.1% | 0.0% | 0.1% | 0.0% |
| | | median | 0.3% | 0.0% | 0.1% | 0.2% | 6.1% | 0.0% | 0.1% | 0.0% | 0.1% | 0.0% |
| | | Q1 | 0.3% | 0.0% | 0.1% | 0.2% | 6.1% | 0.0% | 0.1% | 0.0% | 0.1% | 0.0% |
| | | min | 0.3% | 0.0% | 0.1% | 0.2% | 6.1% | 0.0% | 0.1% | 0.0% | 0.1% | 0.0% |
| Tumor | 9 | max | 229.6% | 91.9% | 122.0% | 118.2% | 119.4% | 95.5% | 96.8% | 55.9% | 234.0% | 68.6% |
| | | Q3 | 155.4% | 46.6% | 61.2% | 95.8% | 97.8% | 67.2% | 73.0% | 21.6% | 161.9% | 42.4% |
| | | median | 46.4% | 19.2% | 17.6% | 65.6% | 52.8% | 17.6% | 58.9% | 5.1% | 86.1% | 15.9% |
| | | Q1 | 10.9% | 0.3% | 8.9% | 41.9% | 28.1% | 3.8% | 37.7% | 1.7% | 56.5% | 12.1% |
| | | min | 1.4% | 0.1% | 2.0% | 17.2% | 13.9% | 2.0% | 12.6% | 0.5% | 2.8% | 1.2% |

TABLE 30
(Clinical validation result for hepatocellular carcinoma)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 2 | max | 0.2% | 0.2% | 0.1% | 0.8% | 12.9% | 0.2% | 0.0% | 0.0% | 1.1% | 0.1% |
| | | Q3 | 0.2% | 0.2% | 0.1% | 0.6% | 12.1% | 0.1% | 0.0% | 0.0% | 0.9% | 0.0% |
| | | median | 0.1% | 0.1% | 0.0% | 0.4% | 11.3% | 0.1% | 0.0% | 0.0% | 0.6% | 0.0% |
| | | Q1 | 0.1% | 0.1% | 0.0% | 0.2% | 10.4% | 0.0% | 0.0% | 0.0% | 0.4% | 0.0% |
| | | min | 0.0% | 0.0% | 0.0% | 0.0% | 9.6% | 0.0% | 0.0% | 0.0% | 0.1% | 0.0% |
| Tumor | 8 | max | 11.8% | 0.2% | 0.7% | 2.1% | 87.2% | 1.1% | 34.5% | 3.4% | 24.6% | 0.2% |
| | | Q3 | 1.5% | 0.0% | 0.4% | 1.0% | 60.8% | 0.1% | 5.6% | 1.7% | 2.8% | 0.0% |
| | | median | 0.1% | 0.0% | 0.3% | 0.5% | 35.7% | 0.0% | 2.7% | 0.1% | 1.5% | 0.0% |
| | | Q1 | 0.0% | 0.0% | 0.1% | 0.0% | 18.8% | 0.0% | 2.0% | 0.0% | 0.9% | 0.0% |
| | | min | 0.0% | 0.0% | 0.0% | 0.0% | 17.2% | 0.0% | 0.0% | 0.0% | 0.2% | 0.0% |

TABLE 31
(Clinical validation result for ovarian cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 2 | max | 0.0% | 0.0% | 0.2% | 0.3% | 1.9% | 0.0% | 0.4% | 0.2% | 0.4% | 0.0% |
| | | Q3 | 0.0% | 0.0% | 0.1% | 0.2% | 1.9% | 0.0% | 0.3% | 0.1% | 0.4% | 0.0% |
| | | median | 0.0% | 0.0% | 0.1% | 0.1% | 1.8% | 0.0% | 0.2% | 0.1% | 0.4% | 0.0% |
| | | Q1 | 0.0% | 0.0% | 0.0% | 0.1% | 1.8% | 0.0% | 0.1% | 0.0% | 0.4% | 0.0% |
| | | min | 0.0% | 0.0% | 0.0% | 0.0% | 1.8% | 0.0% | 0.0% | 0.0% | 0.4% | 0.0% |
| Tumor | 8 | max | 135.1% | 17.7% | 107.0% | 0.0% | 112.4% | 0.1% | 86.7% | 14.1% | 103.9% | 42.0% |
| | | Q3 | 30.2% | 0.4% | 51.9% | 0.0% | 97.9% | 0.0% | 22.8% | 0.5% | 4.4% | 0.0% |
| | | median | 0.1% | 0.0% | 0.2% | 0.0% | 78.7% | 0.0% | 1.1% | 0.0% | 0.3% | 0.0% |
| | | Q1 | 0.0% | 0.0% | 0.0% | 0.0% | 54.3% | 0.0% | 0.1% | 0.0% | 0.1% | 0.0% |
| | | min | 0.0% | 0.0% | 0.0% | 0.0% | 24.9% | 0.0% | 0.0% | 0.0% | 0.0% | 0.0% |

TABLE 32
(Clinical validation result for pancreatic cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 1 | max | 0.3% | 0.4% | 0.7% | 0.8% | 44.5% | 0.6% | 0.1% | 0.0% | 0.4% | 0.0% |
| | | Q3 | 0.3% | 0.4% | 0.7% | 0.8% | 44.5% | 0.6% | 0.1% | 0.0% | 0.4% | 0.0% |
| | | median | 0.3% | 0.4% | 0.7% | 0.8% | 44.5% | 0.6% | 0.1% | 0.0% | 0.4% | 0.0% |
| | | Q1 | 0.3% | 0.4% | 0.7% | 0.8% | 44.5% | 0.6% | 0.1% | 0.0% | 0.4% | 0.0% |
| | | min | 0.3% | 0.4% | 0.7% | 0.8% | 44.5% | 0.6% | 0.1% | 0.0% | 0.4% | 0.0% |
| Tumor | 9 | max | 159.3% | 49.6% | 85.0% | 127.9% | 122.1% | 82.2% | 273.2% | 59.4% | 161.8% | 5.3% |
| | | Q3 | 1.7% | 15.2% | 27.0% | 30.0% | 112.1% | 47.2% | 50.6% | 5.4% | 105.3% | 3.6% |
| | | median | 0.0% | 4.5% | 5.1% | 21.1% | 36.7% | 28.3% | 44.0% | 2.9% | 89.4% | 0.0% |
| | | Q1 | 0.0% | 0.1% | 0.1% | 11.0% | 29.5% | 0.8% | 15.4% | 0.0% | 31.9% | 0.0% |
| | | min | 0.0% | 0.0% | 0.0% | 0.0% | 15.4% | 0.0% | 0.0% | 0.0% | 0.3% | 0.0% |

TABLE 33
(Clinical validation result for prostate cancer)

| Tissue status | n | quartile | ADHFE1 | ADARB2 | EFS | ADAMTS5 | MMP23B | PLD5 | MIR129-2 | IRF4 | NRG1 | KCNQ5 |
|---|---|---|---|---|---|---|---|---|---|---|---|---|
| Normal | 4 | max | 0.5% | 0.1% | 1.8% | 0.7% | 4.4% | 0.0% | 0.2% | 0.1% | 0.6% | 0.4% |
| | | Q3 | 0.2% | 0.0% | 1.0% | 0.6% | 3.1% | 0.0% | 0.1% | 0.0% | 0.5% | 0.4% |
| | | median | 0.1% | 0.0% | 0.6% | 0.3% | 2.7% | 0.0% | 0.0% | 0.0% | 0.3% | 0.3% |
| | | Q1 | 0.0% | 0.0% | 0.4% | 0.1% | 2.4% | 0.0% | 0.0% | 0.0% | 0.1% | 0.2% |
| | | min | 0.0% | 0.0% | 0.3% | 0.0% | 1.6% | 0.0% | 0.0% | 0.0% | 0.0% | 0.0% |
| Tumor | 6 | max | 234.5% | 0.1% | 258.1% | 84.8% | 94.2% | 1.7% | 143.2% | 17.8% | 80.6% | 401.1% |
| | | Q3 | 24.5% | 0.0% | 154.3% | 8.2% | 77.5% | 0.1% | 43.2% | 1.4% | 16.2% | 61.3% |
| | | median | 0.5% | 0.0% | 70.4% | 0.3% | 52.0% | 0.0% | 12.9% | 0.0% | 3.8% | 32.1% |
| | | Q1 | 0.1% | 0.0% | 37.5% | 0.1% | 21.1% | 0.0% | 3.2% | 0.0% | 0.1% | 1.0% |
| | | min | 0.0% | 0.0% | 29.9% | 0.0% | 6.5% | 0.0% | 1.7% | 0.0% | 0.0% | 0.3% |

It shall be appreciated that, in the specification and the claims of the present invention, some terms (e.g., data sets, database, predetermined rule, predetermined threshold, candidate biomarker, difference value, confusion matrix) are preceded by “first,” “second,” “third,” “fourth,” “fifth,” or “sixth.” Please note that “first,” “second,” “third,” “fourth,” “fifth,” and “sixth” are used only for distinguishing different terms. If the order of these terms is not specified or cannot be derived from the context, the order of these terms is not limited by the preceded “first,” “second,” “third,” “fourth,” “fifth,” and “sixth.”

Furthermore, it shall be appreciated that the aforesaid normal subjects and the normal subject group may have different meaning in different embodiments. For example, if the methylation biomarker selection apparatus or method aims to find out the candidate biomarkers and/or target biomarker(s) for a specific race, the aforesaid normal subjects and the normal subject group may be narrowed down to related to subjects of that specific race and without the target disease.

According to the above descriptions, the methylation biomarker selection technique (at least comprises the methylation biomarker selection apparatuses and methods) provided by the present invention utilizes two different kinds of data sets (i.e., the first data sets and the second data sets) to discover candidate biomarkers pertaining to a target disease. While the first data sets comprise methylation degrees of various methylation loci, the second data sets comprise medical record(s). With the first data sets, differentiable loci can be identified as the primary biomarkers pertaining to the target disease. With the second data sets, comorbidities of the target disease, and associated genes thereof can be identified so as to provide the secondary biomarkers pertaining the target disease. As both methylation degrees and comorbidities of the target disease are considered, the methylation biomarker selection technique of the present invention can provide candidate biomarkers that are highly sensitive and highly specific to the target disease. Furthermore, as the candidate biomarkers are determined based on a correlation analysis of the primary biomarkers and the secondary biomarkers, a sufficient amount of candidate biomarkers can be provided.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A methylation biomarker selection apparatus, comprising:
a storage, being configured to store a plurality of first data sets and a plurality of second data sets, wherein each of the first data sets comprises a plurality of methylation degrees corresponding to a plurality of methylation loci, and each of the second data sets comprises at least one medical record; and
a processor, being electrically connected to the storage and configured to perform the following operations:
(a) determining a plurality of primary biomarkers by identifying a plurality of differentiable loci from the methylation loci according to the methylation degrees,
(b) determining a plurality of secondary biomarkers by identifying a plurality of comorbidities of a target disease, and associated genes thereof based on the second data sets, and
(c) determining a plurality of candidate biomarkers based on a correlation analysis of the primary biomarkers and the secondary biomarkers.

2. The methylation biomarker selection apparatus of claim 1, wherein the processor further performs the following operations:
(d) clustering the candidate biomarkers into a plurality of functional clusters,
(e) calculating a weight for each of the candidate biomarkers in each of the functional clusters, and
(f) determining at least one target biomarker from at least one of the functional clusters according to the weights in each of the functional clusters.

3. The methylation biomarker selection apparatus of claim 1, wherein the processor determines the primary biomarkers by performing the following operation:
selecting the methylation loci having at least one of an averaged methylation degree difference conforming to a first predetermined rule and a p-value conforming to a second predetermined rule as the differentiable loci,
wherein the differentiable loci are determined as the primary biomarkers.

4. The methylation biomarker selection apparatus of claim 1, wherein the processor determines the secondary biomarkers by performing the following operations:
calculating an association degree indicating relevance to the target disease for each of the distinct diagnosed diseases,
selecting the diagnosed diseases having the association degree conforming to a third predetermined rule as the comorbidities, and
determining a plurality of genes corresponding to the comorbidities as the secondary biomarkers.

5. The methylation biomarker selection apparatus of claim 4, wherein the association degree of each of the distinct diagnosed diseases comprises an odds ratio, a p-value, and a supporting rate.

6. The methylation biomarker selection apparatus of claim 2, wherein the processor is further configured to calculate at least one gene distance by the following operations:
calculating a Gene Ontology (GO) term distance for each of at least one GO term pair between a first candidate biomarker and a second candidate biomarker, and
determining the gene distance between the first candidate biomarker and the second candidate biomarker according to the at least one GO term distance.

7. The methylation biomarker selection apparatus of claim 6, wherein each of the GO term distances is calculated based on an information content distance and a Czekanowski-Dice distance.

8. The methylation biomarker selection apparatus of claim 2, wherein the processor is further configured to execute a recurrent neural network comprising an encoder, an attention mechanism, and a decoder, each of a plurality of candidate biomarker sequences belongs to one of a normal subject group and a disease subject group, each of the candidate biomarker sequences corresponds to one of the candidate biomarkers, and the processor calculates the weight for each of the candidate biomarkers in each of the functional clusters by the following operations:
deriving a plurality of normal attention weights from the attention mechanism by inputting the candidate biomarker sequences corresponding to the candidate biomarker and from the normal subject group into the recurrent neural network,
deriving a plurality of disease attention weights from the attention mechanism by inputting the candidate biomarker sequences corresponding to the candidate biomarker and from the disease subject group into the recurrent neural network,
calculating an averaged normal weight by averaging the normal attention weights,
calculating an averaged disease weight by averaging the disease attention weights, and
calculating the weight according to the averaged normal weight and the averaged disease weight.

9. The methylation biomarker selection apparatus of claim 2, wherein the processor further ranks the candidate biomarkers in each of the functional clusters according to the corresponding weights.

10. A methylation biomarker selection method for use in an electronic apparatus, the electronic apparatus storing a plurality of first data sets and a plurality of second data sets, each of the first data sets comprising a plurality of methylation degrees corresponding to a plurality of methylation loci, each of the second data sets comprises at least one medical record, and the methylation biomarker selection method comprising the following steps:
(a) determining a plurality of primary biomarkers by identifying a plurality of differentiable loci from the methylation loci according to the methylation degrees;
(b) determining a plurality of secondary biomarkers by identifying a plurality of comorbidities of a target disease, and associated genes thereof based on the second data sets; and
(c) determining a plurality of candidate biomarkers based on a correlation analysis of the primary biomarkers and the secondary biomarkers.

11. The methylation biomarker selection method of claim 10, further comprising the following step:
(d) clustering the candidate biomarkers into a plurality of functional clusters;
(e) calculating a weight for each of the candidate biomarkers in each of the functional clusters; and
(f) determining at least one target biomarker from at least one of the functional clusters according to the weights in each of the functional clusters.

12. The methylation biomarker selection method of claim 10, wherein the step (a) comprises the following step:
selecting the methylation loci having at least one of an averaged methylation degree difference conforming to a first predetermined rule and a p-value conforming to a second predetermined rule as the differentiable loci,
wherein the differentiable loci are determined as the primary biomarkers.

13. The methylation biomarker selection method of claim 10, wherein the step (b) comprises the following steps:
calculating an association degree indicating relevance to the target disease for each of the distinct diagnosed diseases;
selecting the diagnosed diseases having the association degree conforming to a third predetermined rule as the comorbidities; and
determining a plurality of genes corresponding to the comorbidities as the secondary biomarkers.

14. The methylation biomarker selection method of claim 13, wherein the association degree of each of the distinct diagnosed diseases comprises an odds ratio, a p-value, and a supporting rate.

15. The methylation biomarker selection method of claim 11, further comprises the following steps:
calculating at least one gene distance, comprising the following steps:
calculating a GO term distance for each of at least one GO term pair between a first candidate biomarker and a second candidate biomarker; and
determining the gene distance between the first candidate biomarker and the second candidate biomarker according to the at least one GO term distance.

16. The methylation biomarker selection method of claim 15, wherein each of the GO term distances is calculated based on an information content distance and a Czekanowski-Dice distance.

17. The methylation biomarker selection method of claim 11, wherein the electronic apparatus executes a recurrent neural network comprising an encoder, an attention mechanism, and a decoder, each of a plurality of candidate biomarker sequences belongs to one of a normal subject group and a disease subject group, each of the candidate biomarker sequences corresponds to one of the candidate biomarkers, and the step (e) comprises the following steps:
deriving a plurality of normal attention weights from the attention mechanism by inputting the candidate biomarker sequences corresponding to the candidate biomarker and from the normal subject group into the recurrent neural network;
deriving a plurality of disease attention weights from the attention mechanism by inputting the candidate biomarker sequences corresponding to the candidate biomarker and from the disease subject group into the recurrent neural network;
calculating an averaged normal weight by averaging the normal attention weights;
calculating an averaged disease weight by averaging the disease attention weights; and
calculating the weight according to the averaged normal weight and the averaged disease weight.

18. The methylation biomarker selection method of claim 11, further comprising the following step:
ranking the candidate biomarkers in each of the functional clusters according to the corresponding weights.
